(19) United States
(12) Patent Application Publication
(10) Pub. No.: US 2012/0070374 A1
(43) Pub. Date: Mar. 22, 2012

(54) Compounds for Non-Invasive Measurement of Aggregates of Amyloid Peptides

(76) Inventors: Henriksen Gjermund, Starnberg (DE); Behrooz Hooshyar Yousefi, Haar (DE); Hans-Juergen Wester, Ilmmuenster (DE)

(21) Appl. No.: 13/148,076

(22) PCT Filed: Feb. 11, 2010
(86) PCT No.: PCT/EP10/51704

§ 371 Date: Nov. 17, 2011

(30) Foreign Application Priority Data
Feb. 11, 2009 (EP) 09001902.7

(51) Int. Cl. A61K 51/04; G01N 33/566; C12Q 1/02; C07D 513/04

(52) U.S. Cl. 424/1.89; 548/151; 544/238; 424/1.81; 435/7.1; 435/29

(57) ABSTRACT

The invention relates to the provision of compounds, methods for producing them, and their use for imaging and quantification of aggregates of β-amyloid peptides in vivo. In a preferred aspect of the invention, a tracer is administered to humans and displays enrichment in body parts that are containing aggregates of amyloid peptides. Tracers of the invention can be used for non-invasive depiction and quantification of aggregates of β-amyloid peptides in humans affected with diseases that are characterized in the generation of such aggregates.

### BACKGROUND OF THE INVENTION

The prevalence of dementia, particularly Alzheimer's dementia (AD) is increasing with increasing life expectancy. Up to ˜50% of persons>85 years are suffering from AD, thus AD is one of the most common age-related disorders. Even though the causality of pathological processes underlying the development of dementias are not fully understood, growing evidence support the assumptions of a common basis of several neurodegenerative disorders, characterized by an increased production, misfolding and aggregation of peptides or proteins.

The major constituents of deposits of β-amyloid in AD brain are peptides consisting of 40 and 42 amino acids, Aβ₄₀ and Aβ₄₂, generated from the cleavage of amyloid precursor protein. The subsequent arrangement of these peptide monomers includes generation of fibrils that are possessing a characteristic 3-plated sheet structure. There is growing evidences for β-amyloid peptides being essential for modulation of synaptic activity and neuronal survival under normal conditions (Pearson and Peers J. Physiol. 2006; 575:5-10). Based on familial cases, in which point-mutations in the amyloid precursor protein are seen to increase amyloidosis, the formation and aggregation of amyloid peptides have been identified as critical factors in the initiation and progress of AD.

Above a certain critical concentration, Aβ40 and Aβ42 peptides apparently misfold and aggregate. Aβ₄₂ shows stronger aggregative tendency. Depending on density, configuration and constitution, different types of plaques have been described and in general, plaques with a lower proportion of fibrillar components (diffuse plaques) and a higher proportion of fibrillation (mature, dense core, classic or neuritic, or compact plaques) can be differentiated (Dickson and Vikers Neuroscience. 2001; 105:99-107). Recent studies indicate that not the entire Aβ plaques, but possibly smaller Aβ oligomers represent the responsible noxious species, leading to synaptic dysfunction. Several recent studies support the hypothesis of a loss of function, implying that not just a general overproduction of Aβ is the initial trigger of the cascade of AD pathology but rather a imbalance between Aβ₄₀ and Aβ₄₂ amyloid peptides (Van Broeck et al. Neurodegener Dis. 2007; 4:349-65). It has been shown that Aβ₄₂ shows stronger aggregative tendency and preferentially assembles to noxious oligomers, representing the more toxic agents (Yan Y, Wang J Mol. Biol. 2007; 369:909-16; Kim et al. J. Neurosci. 2007; 27:627-33). Some familiar forms of AD the level of Aβ₄₀ is decreased whereas the level of Aβ₄₂ remains stable. Recently, it has been discussed that Aβ₄₂ may actually exert an inhibitory function on presenilin function [Shen and Kelleher Proc Natl Acad Sci USA. 2007; 104:403-9.

β-Amyloid induced neurotoxicity has been proposed to induce neurotoxicity by different mechanisms, including free radical generation (Hensley et al. Proc. Natl. Acad. Sci. USA 1994; 91: 3270-3274) oxidative stress (Van et al. Nature 1996; 382: 685-69,115) or inflammation (Miklossy et al. J. Neuropathol. Exp. Neural. 1999; 58: 803-814). Biochemical and histopathological investigations have revealed that both early-onset familial AD and sporadic AD are characterized in an increased production of Aβ₄₂ (Citron et al. Nat Med 1997; 3:67-72; Mann et al. Ann Neurol 1996; 40:149-156; Kumar-Singh et al. Human mutation 2006; 27; 686-695, 2006) and up to two fold higher concentration of Aβ₄₂ relative to Aβ₄₀ in Aβ-aggregates (Kumar-Singh et al. Human mutation 2006; 27; 686-695). This has led to the Aβ₄₂ seed-hypothesis setting forth that Aβ₄₂ is the major driving force in providing Aβ-aggregates and thus neurotoxicity.

Historically, a definite diagnosis of AD has been obtained by neuropathological investigations of the brain from AD patients post mortem, depicting the presence of senile plaques containing aggregates of β-amyloid (Aβ). Current efforts to a vital method of AD diagnosis include determination of the presence of genes identified to represent an increased risk of developing the disease, determination of the level of β-amyloid peptides in the spinal fluid and in vivo imaging techniques.

New biomarkers suitable for in vivo imaging of in vivo β-amyloid plaque may have the potential to identify neurodegeneration ahead of manifest dementia. In addition to diagnosis, molecular imaging may play a relevant role in treatment selection and monitoring. In neurodegenerative diseases, such as e.g. AD, several modern treatment strategies are directed towards prevention or reversal of peptide deposition in the brain (e.g. β- and γ-secretase inhibitors, vaccination strategies) (Citron Nat Rev Neurosci. 2004; 5: 677-85). Imaging may represent the most valuable surrogate marker particularly with regard to the well-known intraindividual fluctuations of cognitive performance.

An in vivo imaging technique would potentially be useful to determine the presence and the concentration of peptide aggregates at a pre-symptomatic stage. In especially, for providing a method for diagnosing AD over other neurodegenerative disorders as well as monitoring therapeutic effects directed at prevention or reversal of Aβ-plaque deposition in the brain.

In addition to β-amyloid peptides in Alzheimer's disease, aggregates of β-amyloid peptides has been found in other diseases such as type II diabetes, Down's syndrome, Creutzfeldt-Jacob disease, prion mediated diseases, amyloid polyneuropathy and cerebrovascular amyloid angiopathy

### SUMMARY OF THE INVENTION

The present invention provides compounds and tracers thereof for use as in vivo imaging probes in the depiction and quantification of aggregates of β-amyloid peptides. In a preferred embodiment, a tracer of the invention is administered to humans and is enriched in the aggregates of β-amyloid peptides, for example such contained in the brain of patients affected with AD, and is useful in discriminating between diseased individuals affected with aggregates of β-amyloid peptides and a normal individual. In another preferred aspect, radioactive analogues of the compounds of the invention can be obtained in a high yield and specific activity and have a low toxicity at an effective administered amount, including an amount being useful for imaging and quantification of aggregates of β-amyloid peptides or a disease state related to generation of aggregates of β-amyloid peptides. For example a tracer in the invention can be applied for depiction and quantification of aggregates of β-amyloid peptides, or aggregates of tau protein or α-synuclein.

The present invention relates to the provision of compounds that are binding to aggregates of β-amyloid peptides or intermediate stages of β-amyloid peptide polymers, parts or fragments of amyloid β-peptide polymers, in addition to aggregates constituted of β-amyloid peptides and other amyloidogenic peptide or protein with high sensitivity. Tracers of the invention can be used for a non-invasive depiction and quantification of aggregates of β-amyloid peptides in individuals affected with diseases that are characterized by the presence of such aggregates.

In one aspect, the invention relates to a tracer for targeting aggregates of amyloid peptides in vitro or in vivo, said tracer comprising the structure

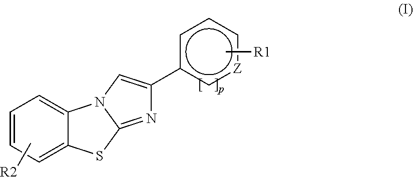

wherein R₁ is selected from the group consisting of H, F, Cl, Br, I, CN, CF₃, alkyl, heteroaryl, heterocycloalkyl and NHR₃, with R₃ being an alkyl, R₂ is —O—R₄, wherein R₄ is selected from the group consisting of CₙH₂ₙ+1, CₙH₂ₙ CₙH₂ₙ-halo, —CH₂—CH═CH-halo and, —[CH₂—CH₂—O]ₘ—[CH₂—CH₂]ₒ-halo, in which halo can be any halogen, preferably wherein halo is F, and with n being in the range of from 1 to 5, with m being in the range of from 1 to 3 and with o being 1, and wherein Z is selected from the group consisting of S, O, N, NH or CH, and wherein p is 0 or 1, wherein the tracer comprises at least one F.

The compounds and tracers of the invention can bind to and detect aggregates of amyloid peptides. In a preferred aspect of the invention, tracers of the invention comprise a radionuclide including for example a photon emitting or a positron emitting radionuclide. For example a label can replace any substituent of a compound of the invention or be an additional substituent in a tracer. In one aspect, a tracer of the invention can comprise one or more of ³H, ¹¹C, ¹⁸F, ¹²³I, ¹²⁴I, ⁷⁵Br, ⁷⁶Br. Most preferably the at least one F atom is ¹⁸F.

The present invention also relates to a method for preparing compounds and tracers comprising a radionuclide, preferably a positron emitting or a photon emitting radionuclide, which upon administration to subjects can be used for depiction and quantification aggregates of amyloid peptides or a characteristic related to generation of amyloid aggregates. Examples of radionuclides include, but are not limited to, ¹¹C, ¹⁸F, ¹²³I, ¹²⁴I, ⁷⁵Br, ⁷⁶Br.

According to a preferred embodiment, the present invention relates to a method for the preparation of a tracer of the general formula

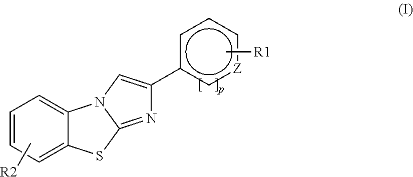

wherein R₁ is selected from the group consisting of H, F, Cl, Br, I, CN, CF₃, alkyl, heteroaryl, heterocycloalkyl and NHR₃, with R₃ being an alkyl, R₂ is selected from the group consisting of —O—CₙH₂ₙ—F, —O—CH₂—CH═CH—F and —O—[CH₂—CH₂—O]ₘ—[CH₂—CH₂]ₒ—F, with F preferably being ¹⁸F, and wherein Z is selected from the group consisting of S, O, N, NH or CH, and wherein p is 0 or 1, by fluorinating a derivative of the general formula II

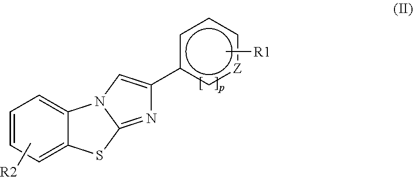

wherein R₁ is selected from the group consisting of H, F, Cl, Br, I, CN, CF₃, alkyl, heteroaryl, heterocycloalkyl and NHR₃, with R₃ being an alkyl, R₂ is selected from the group consisting of —O—CₙH₂ₙ-leaving group, —O—CH₂—CH═CH-leaving group and —O—[CH₂—CH₂—O]ₘ—[CH₂—CH₂]ₒ— leaving group, and wherein Z is selected from the group consisting of S, O, N, NH or CH, and wherein p is 0 or 1, with fluoride, preferably with [¹⁸F]fluoride.

According to another aspect of the invention, the invention relates to the use of tracers of the invention in non-invasive imaging, for instance by means of positron emission tomography (PET) and/or single photon emission tomography (SPECT) and to the use of such compounds in clinical studies in humans and other mammals.

More preferably, the present invention relates to the use of a compound belonging to the invention in the imaging of aggregates of β-amyloid peptides in the central nervous system. The production and presence of one or more pathological proteins, especially amyloid plaques, is encountered in several human diseases and syndromes, including Alzheimer's disease, mild cognitive impairment syndrome, Parkinson's disease, Lewy body dementia, Down's syndrome, frontotemporal degeneration, type II diabetes, Creutzfeldt-Jacob disease, prion mediated diseases, amyloid polyneuropathy and cerebrovascular amyloid angiopathy.

A method of the invention can also include the determination of a localization index of a tracer. In one aspect, the localization index can be the uptake of the tracer in a brain region of interest other than the cerebellum relative to that of cerebellum. In another aspect, the localization index can comprise a comparison of the amount of tracer located in the region of interest of a subject affected with, or suspected to be affected with, a disease related to aggregation of β-amyloid peptides relative to that obtained in a normal control.

### DESCRIPTIONS OF THE DRAWINGS

FIG. 1. Transmission electron microscopy (TEM) images of amyloid aggregates fixated using the negative staining material uranyl acetate. A and B are TEM images of Aβ₄₀ of 33000 and 50000 times magnification, respectively; C and D are TEM images of Aβ₄₂ of 33000 and 50000 times magnification, respectively.

FIG. 2. Inhibition of binding of N—[³H-methyl]6-OH-BTA-1 to fibrils of Aβ40 and Aβ42 from test compounds present at 100 nM.

FIG. 3: Kⱼ values (nM) determined for inhibition of N—[³H-methyl]6-OH-BTA-1 binding to β-amyloid fibrils.

FIG. 4. Brain uptake of selected compounds of the invention and in male Balb-C mice at 5 and 30 min post injection.

FIG. 5. Log P values for selected compounds of the invention.

FIG. 6. Speciation of radioactivity in brain and blood of mice injected with [¹¹C]8 or [¹⁸F]15.

FIG. 7 Regional brain biodistribution of [¹¹C]8 in a PS1/APP double transgenic mouse model of Alzheimer's disease.

FIG. 8. Staining of brain sections of a PS1/APP double transgenic mouse model of Alzheimer's disease with Thioflavin-T (left) and fluorescent antibodies (right).

FIG. 9. Summed PET-images (20-35 min post injection) of [¹¹C]8 in a PS1/APP double transgenic mouse model of Alzheimer's disease of age 16 months (A) and in an age-matched control animal (B).

FIG. 10. Summed PET-images (20-35 min post injection) in a PS1/APP double transgenic mouse model of Alzheimer's disease of age 15.6 months injected with [¹¹C]8 (A) or [¹⁸F]15 (B).

FIG. 11. Left: Ex vivo autoradiography of a transaxial section of the brain of a PS1/APP double transgenic mouse model of Alzheimer's disease killed 40 min after injection of [¹¹C]8. Right: Profile of the activity distribution on the section demonstrating enrichment of activity in plaque containing areas.

FIG. 12. Ex vivo autoradiography of a transaxial section of the brain of an age matched control mouse (A) and in the brain of a PS1/APP mouse (B), both killed 40 min after injection of [¹⁰F]15.

FIG. 13. A: Immunohistochemistry using anti-Aβ antibodies on sections of AD-brain. B: Digital autoradiography of N—[³H-methyl]6-OH-BTA-1 binding to human AD-brain sections incubated in vitro without (i) and with (ii) the presence of compound 8 (left) or compound 15 (right).

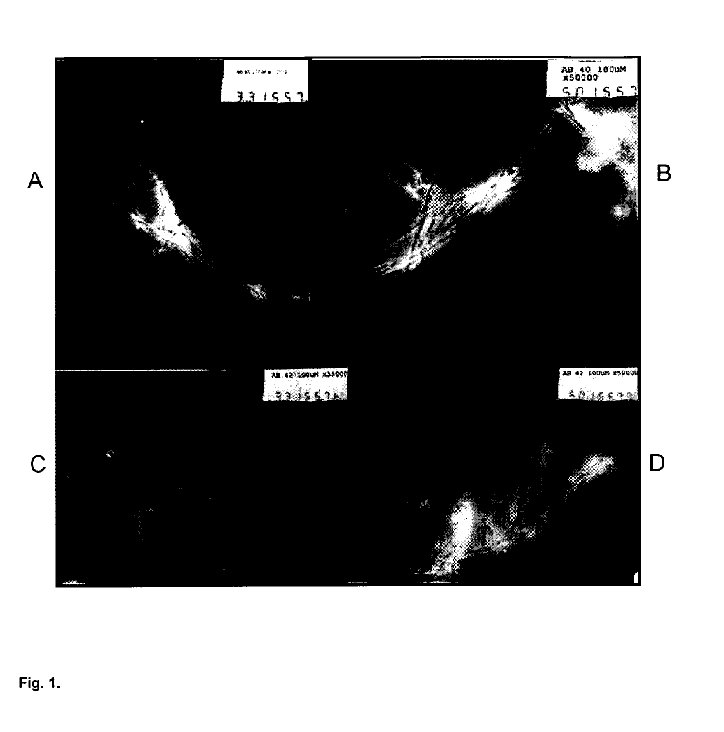

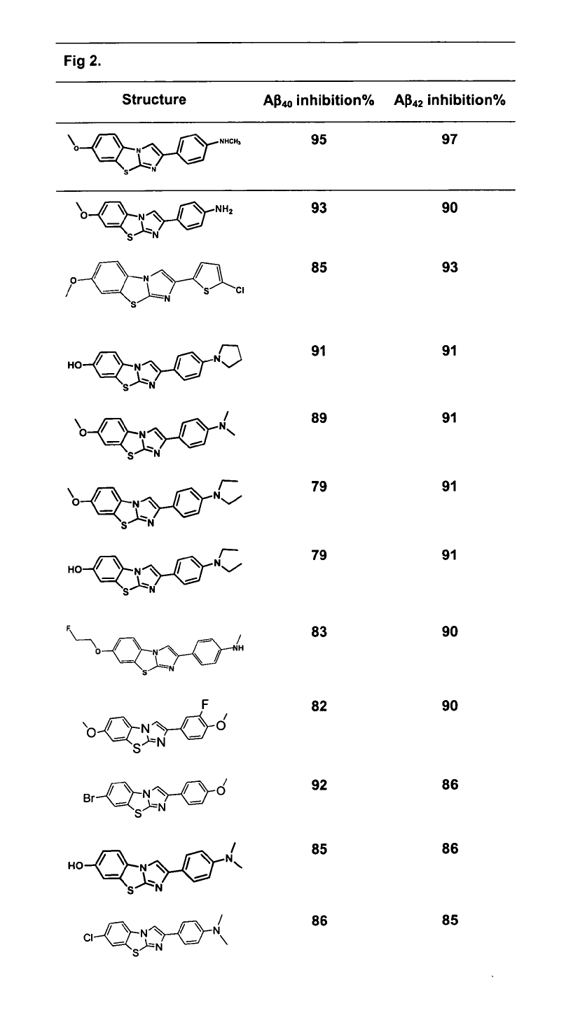

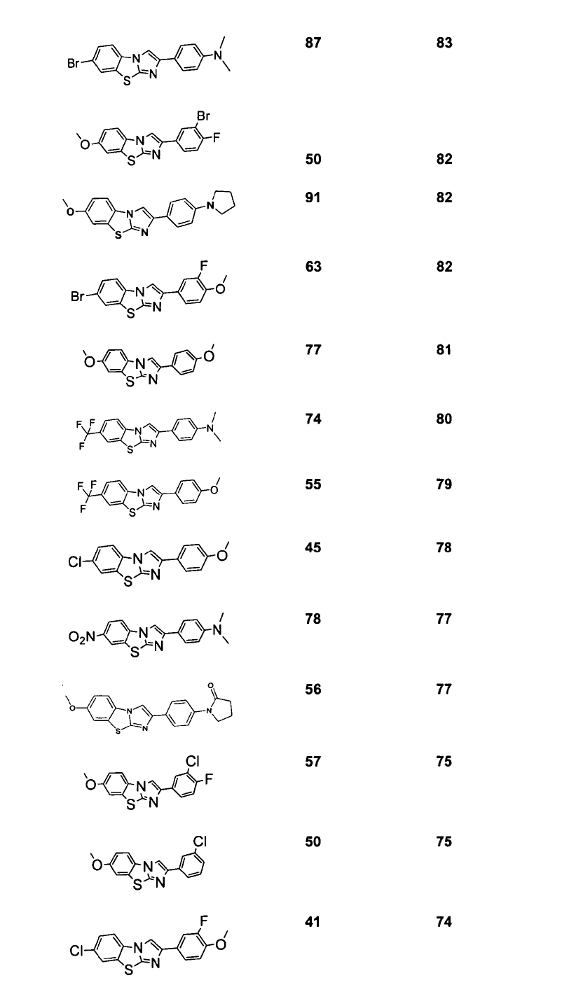

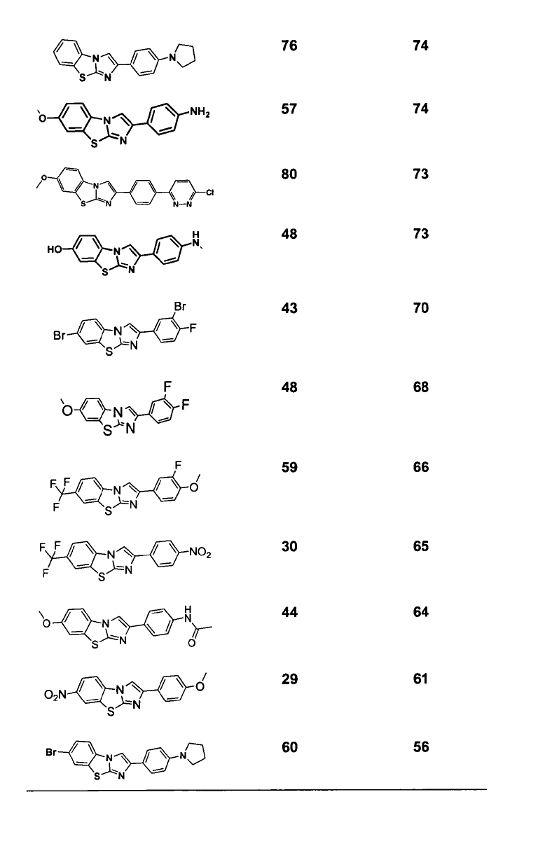

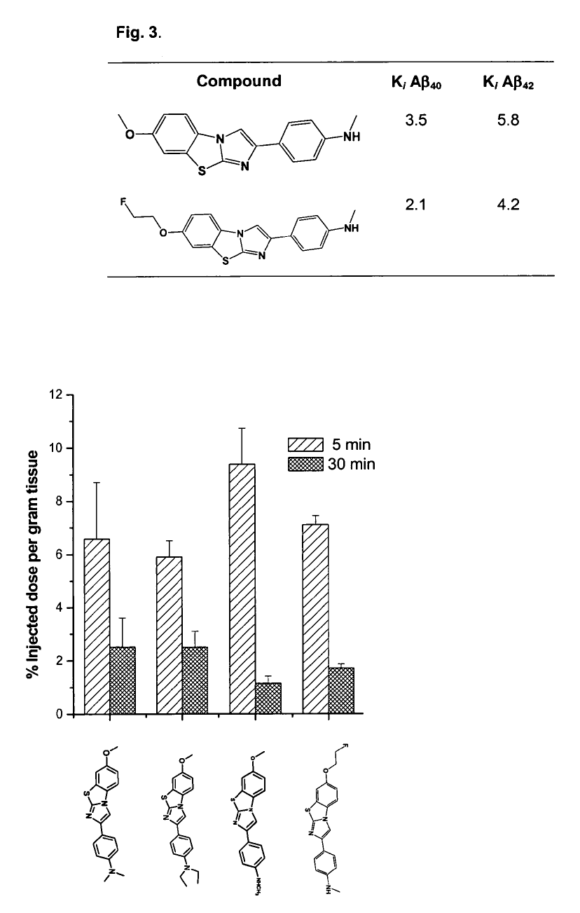

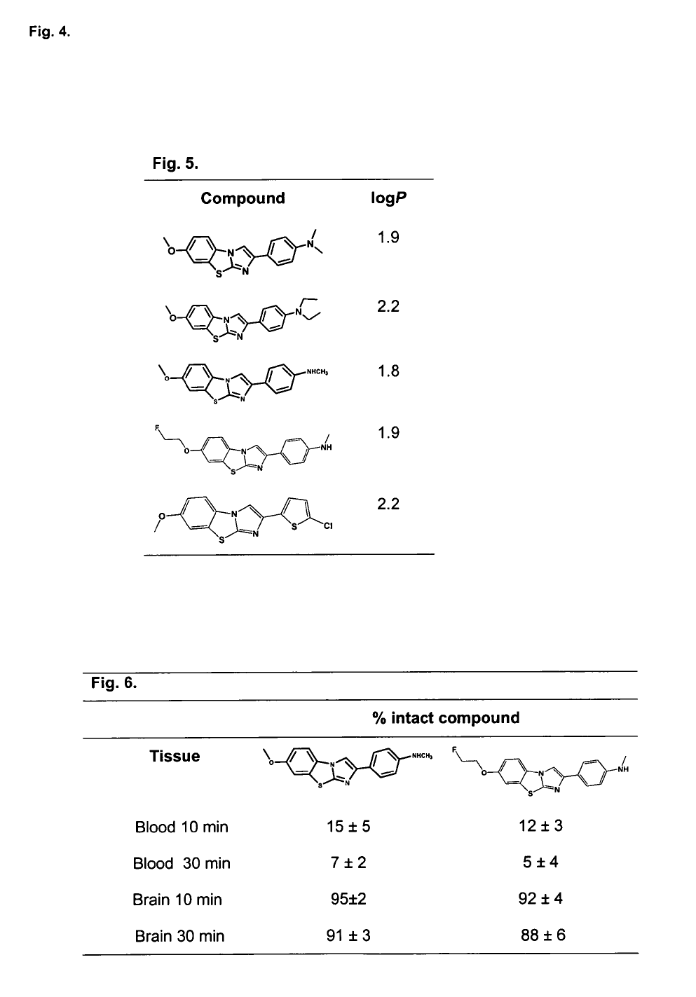

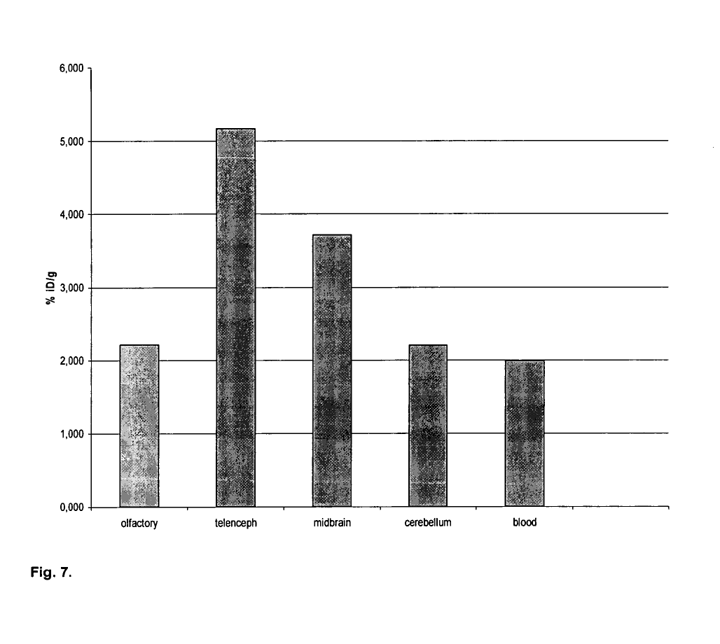

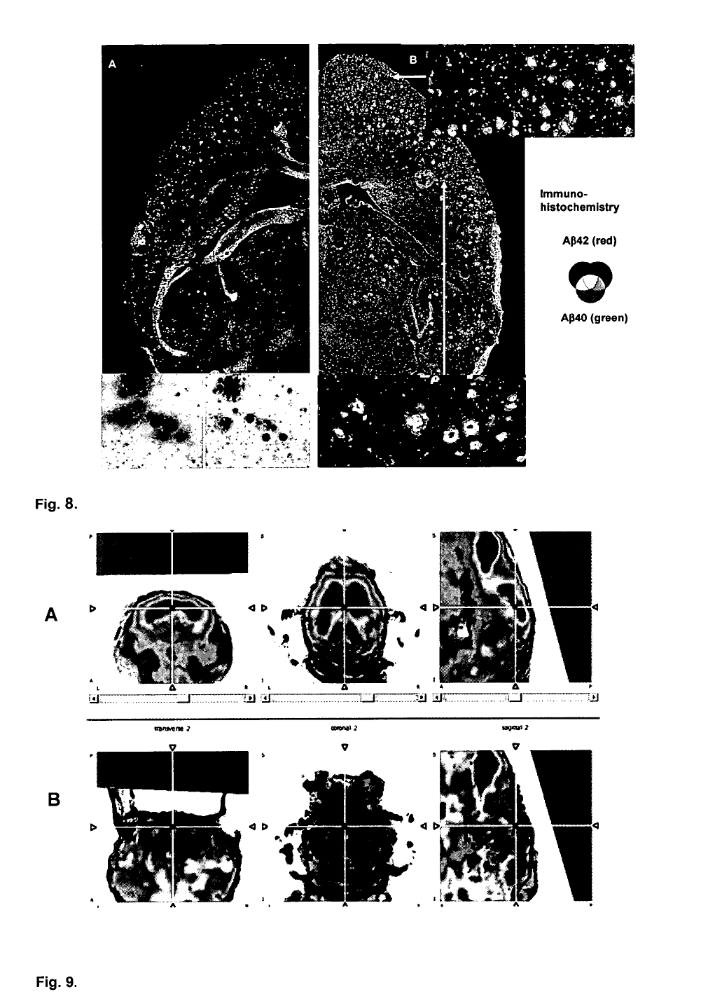

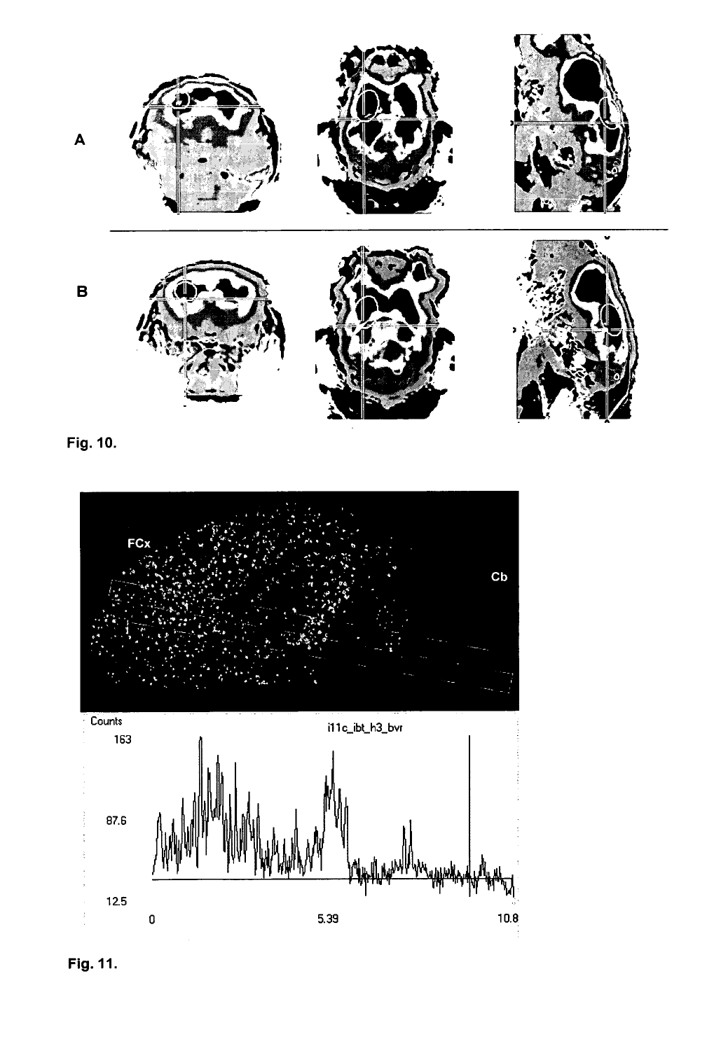

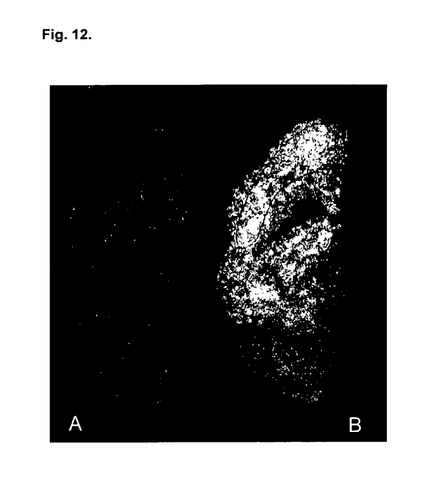

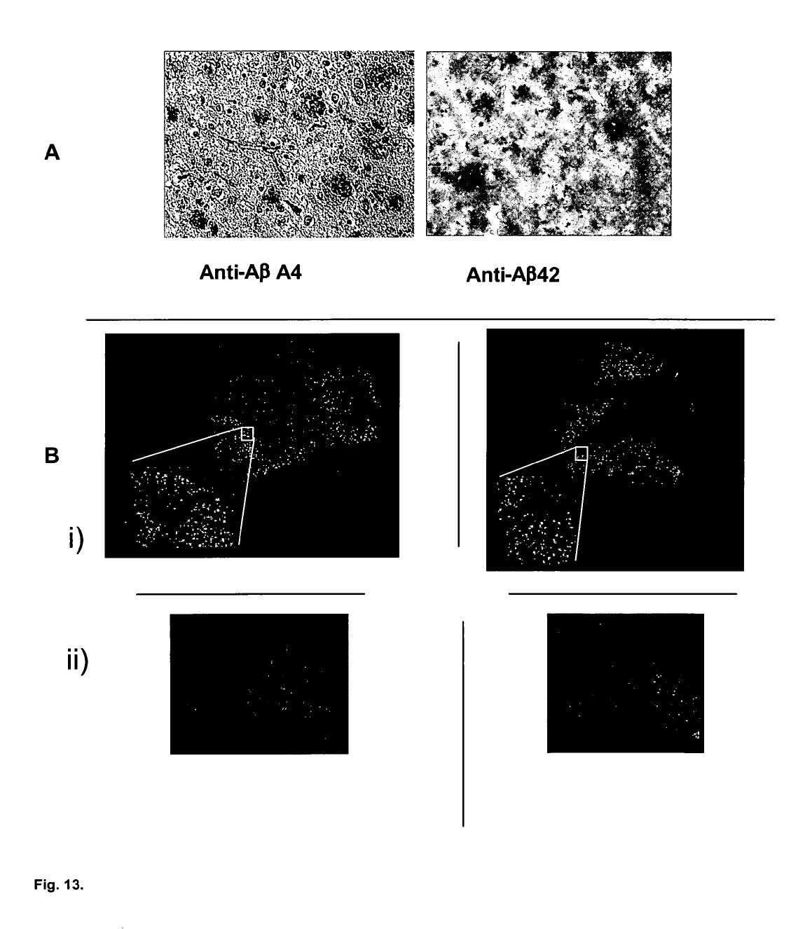

### DETAILED DESCRIPTION OF THE INVENTION

The present invention provides compounds and tracers for use as in vivo imaging probes in the depiction and quantification of aggregates of β-amyloid peptides, for example β-amyloid plaques. In one aspect of the invention a tracer of the invention is administered to humans and is enriched in the aggregates of β-amyloid peptides, for example such contained in the brain of patients affected with AD, useful in discriminating between diseased individuals affected with aggregates of β-amyloid peptides and a normal individual. In another aspect, radioactive analogues of the compounds of the invention can be obtained in a high yield and specific activity and have a low toxicity at an effective administered amount, including an amount being useful for imaging and quantification of aggregates of β-amyloid peptides or a disease state related to generation of aggregates of β-amyloid peptides. Tracers of the invention can be used to depict and quantify aggregates of β-amyloid peptides in individuals affected with diseases such as Alzheimer's disease, mild cognitive impairment syndrome, Parkinson's disease, Lewy body dementia, Down's syndrome, frontotemporal degeneration, type II diabetes, Creutzfeldt-Jacob disease, prion mediated diseases, amyloid polyneuropathy and cerebrovascular amyloid angiopathy.

In one aspect the invention relates to a tracer targeting aggregates of β-amyloid peptides in vitro or in vivo. According to one preferred embodiment of the present invention, the tracers of the invention comprise the structure or formula (I)

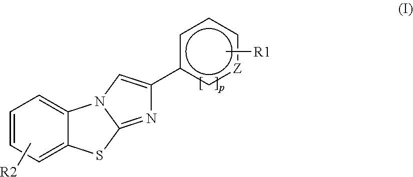

wherein R₁ is selected from the group consisting of H, F, Cl, Br, I, CN, CF₃, alkyl, heteroaryl, heterocycloalkyl and NHR₃, with R₃ being an alkyl, R₂ is —O—R₄, wherein R₄ is selected from the group consisting of CₙH₂ₙ+1, CₙH₂ₙ, CₙH₂ₙ-halo, —CH₂—CH═CH-halo and, —[CH₂—CH₂—O]ₘ—[CH₂—CH₂]ₒ-halo, in which halo can be any halogen, preferably wherein halo is F, and with n being in the range of from 1 to 5, with m being in the range of from 1 to 3 and with o being 1, and wherein Z is selected from the group consisting of S, O, N, NH or CH, and wherein p is 0 or 1, wherein the tracer comprises at least one F, preferably at least one ¹⁸F.

Compared to the use of other radiolabels ¹⁸F has the advantage that it has the relatively long half-life of 109.7 min in contrast to the 20.3 min half-life of ¹¹C, used in most tracers described in the art. This short half life bears several disadvantages, e.g. that such a short half-life constitutes a limiting factor in commercialization since the delivery of compounds having such a short half-life is difficult due to time-consuming transportation. ¹⁸F allows for the production and distribution of radiotracers to nuclear medicine facilities without an on-site cyclotron facility. Further, achievement of a high specific signal of tracer binding to the target often requires monitoring of radioactivity distribution at a time at which the non-specifically bound fraction of tracer has cleared from the target organ or tissue. The half life of 18F is preferential over 11C also due to these requirements.

The herein reported preferred compounds labeled with ¹⁸F overcome these problems in practical use. Further the compounds are useful tracers for detecting amyloid plaques with high sensitivity. This is particularly important in favor of detecting lower levels of amyloid plaques, e.g. in early stages of Alzheimer's disease in living brain,

### The Group Z

Z is selected from the group consisting of S, O, N, NH or CH, and wherein p is 0 or 1.

According to a preferred embodiment, he structural unit

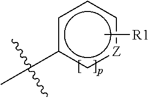

is selected from the group consisting of

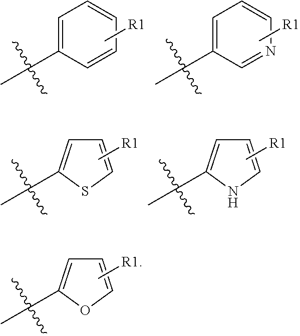

According to a preferred embodiment of the invention, Z is CH and p is 1, the tracer thus having a structure according to the following formula:

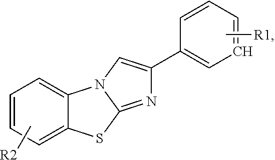

more preferably

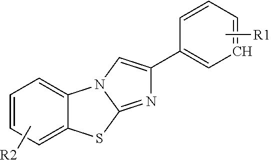

According to an alternative preferred embodiment of the invention, p is 0 and Z is S. In said case, the tracer, accordingly, has a structure according to the following formula:

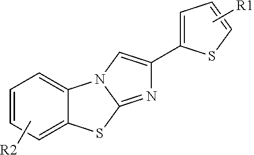

more preferably

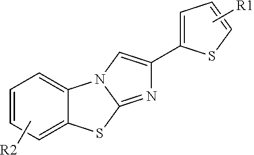

### Residue R₁:

As regards, residue R₁, said residue is selected from the group consisting of H, F, Cl, Br, I, CN, CF₃, alkyl, heteroaryl, heterocycloalkyl and NHR₃, with R₃ being an alkyl.

The term alkyl as used within this application includes any linear, branched or cyclic saturated hydrocarbon. Preferably, the alkyl is a C₁ to C₁₀ alkyl, more preferably a C₁ to C₈ alkyl, more preferably a C₁ to C₆ alkyl, more preferably C₁ to C₃ alkyl, and most preferably methyl.

According to an alternative embodiment, the term alkyl group is denoted to mean an alkyl chain optionally being substituted or being interrupted by heteroatoms like N, O and S. Preferred are interruptions by up to four heteroatoms within the carbon chain. Examples of these interruptions include ethers, thioethers, amines and polyethylene glycols (PEGs). Preferred aliphatic chains with heteroatom interruptions include PEG with 2 to 6 PEG groups and their corresponding polythioether equivalents. In another preferred embodiment, the PEG substituents contain a halogen substitution, preferably a terminal halogen substitution.

Optionally, the alkyl may further be substituted. Substitution include halogen atoms, groups containing heteroatoms like OR, NR₂, SR, SCN, CN, COOR, wherein R is H, alkyl, alkenyl, alkynyl or aryl.

Most preferably, in case R₁ is an alkyl group, said alkyl group is selected from the group consisting of methyl, ethyl, propyl, preferably said alkyl group is methyl.

The term “alkenyl” can refer to an unsaturated straight of branched chain hydrocarbon radical comprising at least one carbon-to-carbon double bond. The chain can be constituted of up to 8 carbons, preferably 5 carbons, more preferably 4 carbons. Examples include, but are not limited to ethenyl, propenyl, iso-propenyl, butenyl, iso-butenyl, t-butenyl. Furthermore, “alkenyl” can refer to an unsaturated straight or branched chain hydrocarbon radical comprising at least one carbon-to-carbon triple bound. The chain can be constituted of up to 8 carbons, preferably 5 carbons, more preferably 4 carbons. Examples include, but are not limited to ethylyl, propynyl, iso-propynyl, butynyl, isobutynyl, t-butynyl. A compound or tracer of the invention can comprise one or more alkenyl or alkynyl substituents.

The term “heterocycloalkyl” as used in the context of the present invention, preferably represent a 4 to 7-membered mono-heterocyclic ring and consist of carbon atoms and from one to three heteroatoms selected from the group consisting of N, O and S. Examples include, but are not limited to piperidinyl, homopiperidinyl, pyrrolyl, pyrrolidinyl, tetrahydrofuranyl.

The term “heteroaryl” as used in the context of the present invention, includes optionally suitably substituted 5- and 6-membered single-ring aromatic groups as well as substituted or unsubstituted multicyclic aryl groups, for example tricyclic or bicyclic aryl groups, comprising one or more, preferably from 1 to 4 such as 1, 2, 3 or 4, heteroatoms, wherein in case the aryl residue comprises more than 1 heteroatom, the heteroatoms may be the same or different. Such heteroaryl groups including from 1 to 4 heteroatoms are, for example, benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl or indolizinyl.

The term “heteroatom” can represent an oxygen atom (“O”), a sulphur atom (“S”) or a nitrogen atom (“N”). It will be recognized that when the heteroatom is nitrogen, it may form an NR′R″ in which R′ and R″ are, independent from another, hydrogen, C₁₋₄ alkyl, C₂₋₄ aminoalkyl, C₁₋₄ haloalkyl or halo benzyl. R′ and R″ can be taken together to form a 5 to 7-member heterocyclic ring that optionally comprises O, S or NR′″ in which R′″ is hydrogen or C₁₋₄ alkyl. A compound or tracer of the invention can comprise one or more heteroatom substituents.

The term “halogen” or “halogen atom” as used in the context of the present invention, preferably refers to a chlorine, iodine, bromine or fluorine atom and the respective radioactive and non-radioactive isotopes. Thus, the term halogen is for example selected from the group consisting of Cl, Br, I, ¹²⁰I, ¹²⁴I, ¹²⁵I, ¹³¹I, ⁷⁵Br, ⁷⁶Br, ⁷⁷Br, ¹⁸F and ¹⁹F and ³⁴Cl. Preferred halogen atoms are fluorine and/or iodine atoms. The term halogen thus also includes the respective Isotopes of these atoms. Thus, in case, the tracer comprises at least one position F, e.g., at least one fluorine atom, the fluorine atom may be selected from the group consisting of ¹⁸F and ¹⁹F. In case, in case, the tracer comprises at least one position F, the F is preferably ¹⁸F.

According to a preferred embodiment, the present invention relates to a tracer as described above, wherein R₁ is selected from the group consisting of NHR₃, a, optionally substituted, nitrogen containing heteroaryl and a, optionally substituted, nitrogen containing heterocycloalkyl

In case R₁ is an NHR₃ and R₃ is an alkyl group, said alkyl group is preferably selected from the group consisting of methyl, ethyl, propyl, in particular said alkyl group is methyl.

Thus, the present invention also describes a tracer as described above, having the structure according to the following formula:

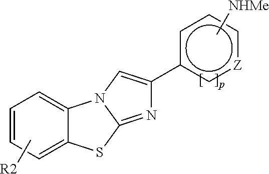

more preferably according to the following formula:

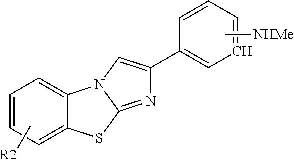

In case the Z is CH and p is 1, R₁ may be located in o-, m- or p-position to the tricyclic ring. Most preferably, in said case, R₁ is located in p-position, thus the tracer has preferably the following formula:

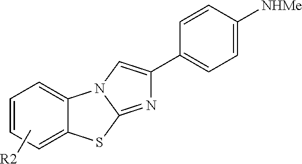

In case R₁ is a, optionally substituted, nitrogen containing heteroaryl, R₁ is preferably selected from the group consisting of, optionally substituted, pyridyl, pyrazinyl, pyriminidyl, pyridazinyl, in particular R₁ is a, optionally substituted, pyridazinyl. Optionally, the heteroaryl may further be substituted. Substitution include halogen atoms, groups containing heteroatoms like OR, N(R)₂, SR, SCN, CN, COOR, wherein R is H, alkyl, alkenyl, alkynyl or aryl. Most preferably, in case R₁ is a substituted heteroaryl, said heteroaryl is substituted with a halogen atom, such as Cl, Br, I or F, in particular with I. According to one embodiment of the present invention R₁ is thus a heteroaryl, preferably pyridazinyl, more preferably a pyradizynl substituted with I.

Thus, the present invention also describes a tracer as described above, having the structure according to the following formula:

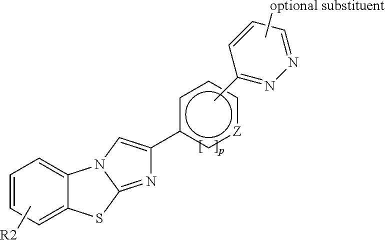

more preferably the following structure

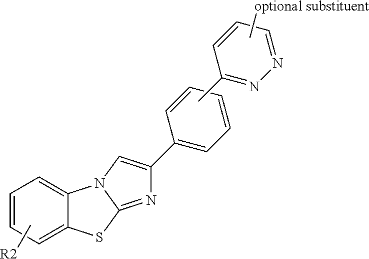

more preferably the following structure

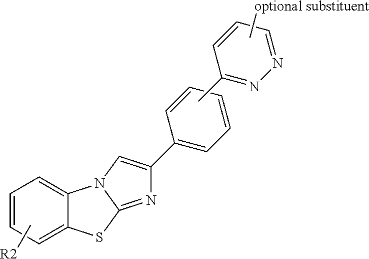

In case R₁ is a, optionally substituted, nitrogen containing heterocycloalkyl, R₁ is preferably selected from the group consisting of, optionally substituted, pyrrolidinyl, piperidinyl and azepanyl. In particular R₁ is a, optionally substituted, piperidinyl. Optionally, the heterocycloalkyl may further be substituted. Substitution include halogen atoms, groups containing heteroatoms like OR, N(R)₂, SR, SCN, CN, COOR, wherein R is H, alkyl, alkenyl, alkynyl or aryl. Most preferably, in case R¹ is a substituted heterocycloalkyl, said heterocycloalkyl is substituted with a halogen atom, such as Cl, Br, I or F, in particular with I. According to a preferred embodiment, case R₁ is a nitrogen containing heterocycloalkyl and said heterocycloalkyl is not further substituted. According to one embodiment of the present invention R₁ is thus piperidinyl.

Thus, the present invention also describes a tracer as described above, having the structure according to the following formula:

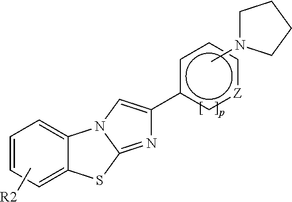

more preferably the following structure

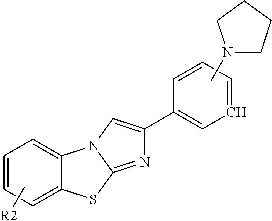

more preferably the following structure

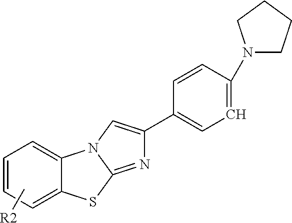

According to a particular preferred embodiment, the present invention relates to a tracer, as described above, wherein R₁ is NHR₃, preferably wherein R₃ is NHMe. Thus, the present invention also relates to a tracer as described above, having the structure according to the following formula:

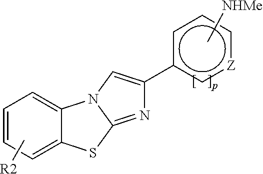

most preferably the following structure

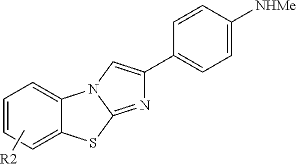

The compound of the invention being substituted with a mono-methylated NH group (R1 NHMe) surprisingly show superior results as regards their binding affinities, lipophilicity and uptake kinetics when compared to compounds bearing an N(Me)₂ group instead. Another specific advantage, in particular compared to compounds bearing an N(Me)₂ group, is their capability of being substituted with O—¹⁸F-fluoroalkyl groups as group R2 with high yields and without significant effect on their binding affinities, lipophilicity and uptake kinetics as further described below and in the examples.

### Residue R₂:

As regards, residue R₂ said residue is —O—R₄, wherein R₄ is selected from the group consisting of CₙH₂+1, CₙH₂ₙ, CₙH₂ₙ-halo, —CH₂—CH═CH-halo and, —[CH₂—CH₂—O]ₘ—[CH₂—CH₂]₀-halo, in which halo can be any halogen, preferably wherein halo is F, and with n being in the range of from 1 to 5, with m being in the range of from 1 to 3 and with o being 1,

Preferably, R₂ is selected from the group consisting of —O—CₙH₂ₙ—F, —O—CH₂—CH═CH—F and —O—[CH₂—CH₂—O]ₘ—[CH₂—CH₂]ₒ—F, preferably wherein F is ¹⁸F.

Thus, the present invention also relates to a tracer as described above, having a structure according to the following formula

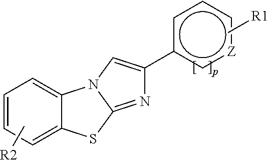

Preferably according to the following formula

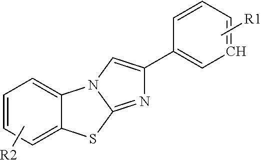

wherein R₂ is selected from the group consisting of —O—CₙH₂ₙ—F, —O—CH₂—CH═CH—F and —O—[CH₂—CH₂—O]ₘ[CH₂—CH₂]ₒ˜F, preferably wherein F is ¹⁸F, and wherein R₂ is preferably located in p-position and, wherein more preferably R₁ is as well located in p-position.

According to a preferred embodiment, R₂ is selected from the group consisting of —O—CH₂CH₂—F, —O—CH₂CH₂CH₂—F, —O—CH₂CH₂—O—CH₂CH₂—F and —O—CH₂CH₂—O—CH₂CH₂—O—CH₂CH₂—F, preferably wherein F is ¹⁸F.

According to a preferred embodiment, R₁ is NHMe and R₂ is —O—CH₂CH₂—F, —O—CH₂CH₂CH₂—F, —O—CH₂CH₂—O—CH₂CH₂—F and —O—CH₂CH₂—O— CH₂CH₂—O—CH₂CH₂—F, preferably R₂ is —O—CH₂CH₂—F, preferably wherein F is ¹⁸F.

Thus, the present invention also relates to a tracer, as described above, said tracer having a structure selected from the group consisting of:

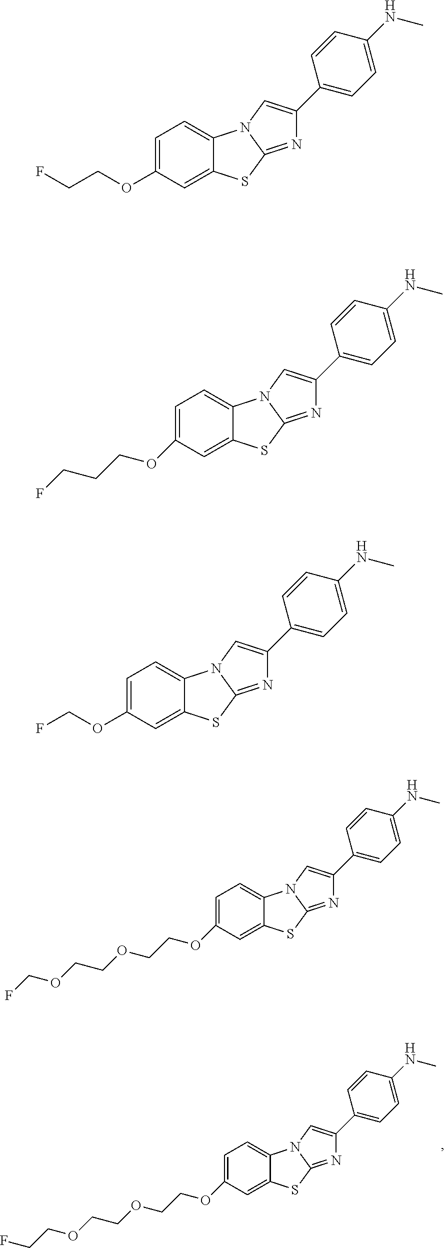

most preferably wherein F is ¹⁸F.

According to a more preferred embodiment, R₂ is —O—CH₂CH₂—F, most preferably wherein F is ¹⁸F, in particular wherein the tracer has the following structure:

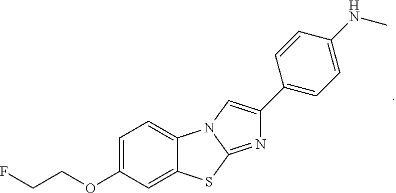

According to an alternative preferred embodiment of the invention, R₂ is —O—CH₂CH₂—F and R1 is a hetroaryl or a heterocycloalkyl, as described above. In said case, the tracer preferably has a structure selected from the following formulas:

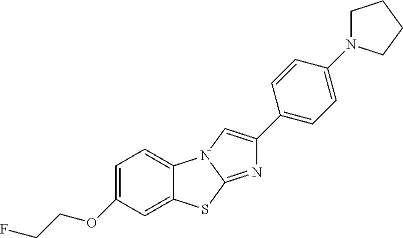

or according the following formula

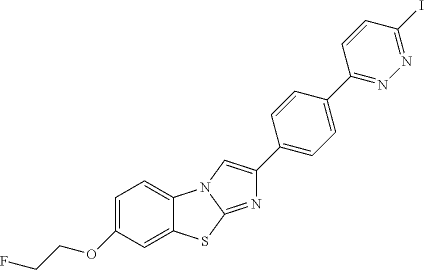

According to an alternative embodiment p is 0 and Z is S, as described above. In said case, the tracer, accordingly, has a structure according to the following formula:

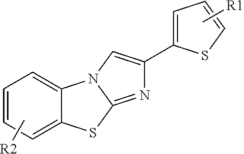

In said case R₁ is preferably a halogene, most preferably I, the tracer thus in particular having the structure:

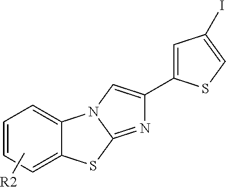

in particular with R₂ being located in p-position.

Further, the following embodiments are encompassed by the present invention, wherein R₂ is CₙH₂ₙ+1 and the fluor atom is not encompassed by R₂ but instead R₁ comprises the at least one fluor atom. In said case, Z is preferably a heteroatom, most preferably S and p is preferably 0. A preferred tracer according to said embodiment comprises the following structure:

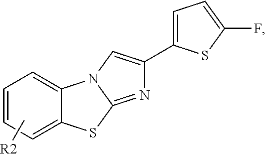

more preferably the following structure

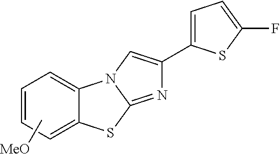

in particular with R₂ being located in p-position.

By way of example, the following preferred compounds of the invention are mentioned:

TABLE 1

| | |
|---|---|
| 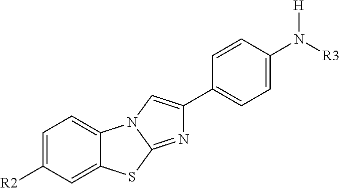 | R₃ = CₙH₂ₙ₊₁, preferably methyl R₂ = OR₄ where R₄ = O—CₙH₂ₙ—F, —O—CH₂—CH═CH—F or —O—[CH₂—CH₂—O]ₘ—[CH₂—CH₂]ₒ—F |
| 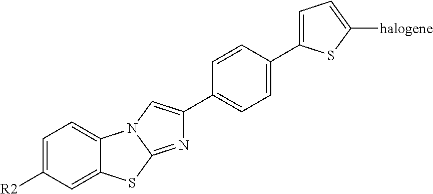 | R₂ = OR₄ where R₄ = O—CₙH₂ₙ—F, —O—CH₂-CH═CH—F or —O—[CH₂—CH₂—O]ₘ—[CH₂—CH₂]ₒ—F and where halogene = F, Cl, Br, I, preferably I. |
| 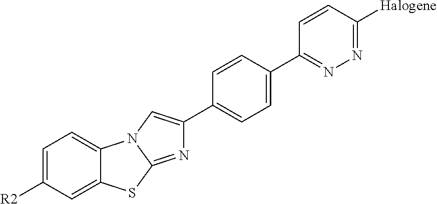 | R₂ = OR₄ where R₄ = O—CₙH₂ₙ—F, —O—CH₂—CH═CH—F or —O—[CH₂—CH₂—O]ₘ—[CH₂—CH₂]ₒ—F and where halogene = F, Cl, Br, I, preferably I |
| 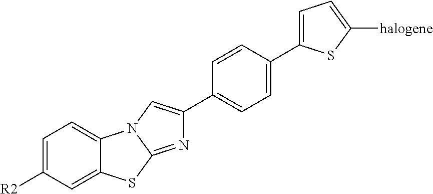 | R₂ = OR₄ where R₄ = CₙH₂ₙ + 1, CₙH₂ₙ, preferably CₙH₂ₙ + 1, more preferably CH₃, and where halogene = F |

As described above, the compounds and tracers of invention bind to and are used to detect aggregates of amyloid peptides. In a preferred aspect of the invention, tracers of the invention comprise a radionuclide including for example a photon emitting or a positron emitting radionuclide. For example a label can replace any substituent of a compound of the invention or be an additional substituent in a tracer. In one aspect, a tracer of the invention can comprise one or more of ³H, ¹¹C, ¹⁸F, ¹²³I, ¹²⁴I, ⁷⁵Br, ⁷⁶Br. More preferably the tracer comprises, as described above, at least one ¹⁸F, in particular wherein the at least one F atom present in the tracer of the invention is an ¹⁸F atom.

In one aspect, compounds and tracers of the invention are used for detection of aggregates of β-amyloid peptides in the body by means of positron emission tomography (PET). In another aspect, compounds and tracers of the invention can be used for detection of aggregates of β-amyloid peptides in the body by means of single photon emission tomography (SPECT). In yet another aspect, given appropriate configuration and constitution of the molecule, the compounds and tracers of the invention can be used for detection of aggregates of β-amyloid peptides in the body by means of PET and SPECT. The compounds and tracers of the invention can be fitted, according to demand, with a positron emitting radionuclide, useful for PET, or alternatively a photon emitting radionuclide, useful for SPECT. In such a manner, an identical chemical compound can be used for PET and SPECT depending on the radionuclide in question.

### Method of Fluorination:

The invention further relates to a method for the preparation of tracers preferably detectable in the body by use of PET and/or SPECT. A compound or tracer of the invention preferably contains at least one radiohalogen, as described above. The radiohalogen can preferably be selected among fluorine, iodine and bromine radioisotopes. For SPECT, the halogens of suitable half-life and gamma energy for biomedical applications include without limitation, ¹²³I (t ½=13 h, Eγ 159 keV), ¹²³I (t ½=8.3 d), and ⁷⁷Br, ⁷⁵Br and ⁷⁶Br.

As regards the method of preparation, bromine and iodine radionuclides can be introduced into a compound of the invention according to methods known in the art (Wilbur. Bioconjug Chem. 1992; 3:433-70; Maziere et al. Radioionidation Reactions for Pharmaceuticals, Springer Verlag, Berlin).

[¹⁸F]Fluoride is one of the most useful PET radionuclides, currently used in clinical nuclear medicine diagnosis. With regard to the nuclear properties, ¹⁸F has a relatively pure (97% probability) decay mode through the emission of a positron (β*) of which the E_{β+max} of 633.5 keV is the lowest of the radionuclides that are commonly used in PET. Consequently, the average range of the positron from ¹⁸F in biological tissues of about 0.3 mm is less than the inherent resolution of clinical PET-scanners. The 109.7 min half-life of ¹⁸F allows for the production and distribution of radiotracers to nuclear medicine facilities without an on-site cyclotron facility and scanning procedures extending several hours.

[¹⁸F]Fluoride is commonly available from cyclotrons on a Ci-scale (1 Ci=37 GBq). Among the four main production routes that have been described in the literature, the ¹⁸O(p,n)¹⁸F nuclear reaction on highly enriched H₂[¹⁸O]O is the most effective one, and gives [¹⁸F]Fluoride in high specific activities (up to about 5000 mCi/μmmol) suitable for nucleophilic reactions of a variety of substrates.

¹⁸F-labeled compounds can be used in positron emission tomography (PET) to allow for the non-invasive imaging of, inter alia, biological structures, particularly those of mammals. This can involve depiction and quantification of receptors and transporters. To be generally useful, a radiopharmaceutical based on a short lived radionuclide such as ¹⁸F needs to be obtainable rapidly in high radiochemical yield and with high specific radioactivity. ¹⁸F has relatively short physical half life of 109.7 min, which means that any process for the manufacture of ¹⁸F-labeled compounds needs to occur in a short time period and has to be capable of providing the desired yields and specific activity of the compounds in question.

The literature comprises many different ways of forming ¹⁸F-labeled compounds although many of these processes involve the use of a precursor for radiolabeling activated for nucleophilic attack. The literature comprises some ways of preparing ¹⁸F-labeled compounds by electrophilic substitution. For preparing ¹⁸F-labeled compounds in a high specific radioactivity (>100 mCi/μmol), this method is hampered by the necessity of introducing carrier fluorine.

The vast majority of fluorination syntheses described in the literature concern the formation of non-radioactive species and the chemistry involved is therefore not applicable as outlined below. The process requirements for the preparation of non-radioactive fluorine compounds are undemanding compared to synthesis of radioactive compounds.

High radiochemical yield is obviously desirable in the same way that high yield is desirable in any organic synthesis. If more ¹⁸F is incorporated into the final product then the amount of active compound formed is higher and there is less wasted expensive starting material.

High specific radioactivity is an important issue in PET studies where the amount of tracer should be minimized to keep the biological system unperturbed. Especially for compounds intended for use in quantifying receptors in the central nervous system (CNS), which is often present at a relatively low concentration, these requirements are often limiting. Thus, these requirements impose significant limitations on the chemical-synthetic procedures used for the preparation of the ¹⁸F-labeled compounds relative to non-radioactive compounds.

A further problem faced during the synthesis of ¹⁸F-labeled compounds is the possibility of side reactions. In conventional non radioactive synthesis, stoichiometric amounts of reactants would be used. This is often not possible in ¹⁸F-compound synthesis and there is typically considerable molar excess of reagents and reactants than the ¹⁸F-labeled species. The difference in the molar ratio of the ¹⁸F-containing reagent relative to other reagents present in the reaction mixture (including the molecule or species to be radiolabeled, called the precursor for radiolabeling) can represent an important difference to procedures in which the stoichiometry of reagents is in the range normally encountered in the synthesis of non-radioactive compounds. In particular, due to the relatively low amount of ¹⁸F-containing reactant present, impurities in the reaction mixture can be an important loss of yield if they possess a comparable or higher reactivity than the precursor for radiolabeling. Thus, side-reactions that are of low importance for a high yield of a given product in a non-radioactive synthesis can be a limiting factor for ¹⁸F-radiolabeling reactions.

A further issue with radiosyntheses involving ¹⁸F is the potential exposure of the chemist to radiation. ¹⁸F syntheses of radiopharmaceuticals for use in PET are typically performed in lead shielded boxes (called hot-cells) and the manipulation of reagents, reactants and equipment is performed via a remote controlled apparatus (called the syntheses module). Under such conditions, the number of synthetic steps, the overall length of the procedure performed with the radioactivity present greatly affects the performance, including reliability and reproducibility, of the procedure. It is therefore advantageous to have as few steps in the procedure as possible, with the highest possible radiochemical yield in order to ensure a high performance of the radiosynthesis and to reduce the complexity of the apparatus required.

In nucleophilic ¹⁸F-fluorinations, starting with no-carrier-added, [¹⁸F]fluoride is the sole suitable approach for preparing ¹⁸F-labeled compounds of a high specific activity. Due to the high charge density, the fluoride ion is highly solvated in aqueous solution and a relatively poor nucleophile. As a strong base, it is protonated in the presence of acidic protons to form H[¹⁸F]F. Consequently, several major aspects have to be considered in order to reach a nucleophilic ¹⁸F-fluorination that is suitable for preparative procedure in radiopharmaceutical chemistry. Firstly, the fluoride ion has to be activated in order to achieve a useful nucleophilicity and solubility. Often dipolar aprotic solvents are used as the reaction medium in order to avoid protonation of the fluoride. Due to the low molar concentration of reactive ¹⁸F-fluoride encountered at labeling reactions designed for obtaining a high specific activity of the product, potential undesired processes such as absorption losses, chemical side reactions and pseudo-carrier effects and presence of water implies that high chemical purity of chemicals, precursors and solvents are required to obtain a relevant radiochemical yield.

An object of the present invention is to provide a fast method for the preparation of ¹⁸F-labeled compounds wherein the ¹⁸F is introduced in a single step.

A ¹⁸F-labeled compound is preferably a compound comprising the linkage —C—¹⁸F where C is a sp³ hybridized carbon atom. According to the present invention, ¹⁸F is preferably introduced into a molecule containing a leaving group.

Thus, the invention also describes a preferred method for the preparation of a tracer of having the general formula

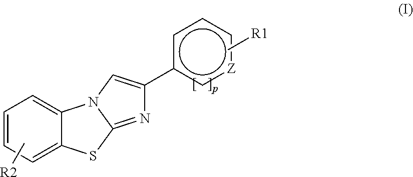

wherein R₁ is selected from the group consisting of H, F, Cl, Br, I, CN, CF₃, alkyl, heteroaryl, heterocycloalkyl and NHR₃, with R₃ being an alkyl, R₂ is selected from the group consisting of —O—CₙH₂ₙ—F, —O—CH₂—CH═CH—F and —O—[CH₂—CH₂—O]ₘ—[CH₂—CH₂]ₒ—F, with F being preferably ¹⁸F, and wherein Z is selected from the group consisting of S, O, N, NH or CH, and wherein p is 0 or 1, by fluorinating a derivative of the general formula II

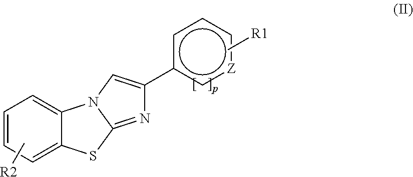

wherein R₁ is selected from the group consisting of H, F, Cl, Br, I, CN, CF₃, alkyl, heteroaryl, heterocycloalkyl and NHR₃, with R₃ being an alkyl, R₂ is selected from the group consisting of —O—CₙH₂ₙ-leaving group, —O—CH₂—CH═CH-leaving group and —O—[CH₂—CH₂—O]ₘ—[CH₂—CH₂]ₒ leaving group, and wherein Z is selected from the group consisting of S, O, N, NH or CH, and wherein p is 0 or 1, with fluoride, preferably [¹⁸F]fluoride.

In the compound (II), i.e. the precursor compound for synthesis of a tracer according to the general formula (I), any leaving group (LG) contained in R₂ may be present, suitable for being substituted by a fluorine. The skilled chemist is aware of numerous leaving groups regularly used in organic synthesis and any of these may be used here. Preferred leaving groups are listed in standard text books such as Jerry March, Advanced Organic Chemistry and include alkoxys, halides and sulphonate esters. Preferred halides are chloride, bromide and iodide. Preferred sulphonate esters are brosylate, nosylate, mesylate, nonaflate, tresylate, triflate or tosylate, especially triflate, tosylate or mesylate.

In a preferred embodiment LG is selected from the group consisting of halogen atoms, tosylsulfonate, trifluoromethanesulfonate, tolyl-sulfonatetriflate, tosylate, p-bromobenzenesulfonate, 4-nitrobenzenesulfonate, trialkyl ammonium, nitro and azide. Preferred halogen atoms are chlorine, bromine and iodine.

Thus, the present invention also relates to a method, as described above, wherein the leaving group is selected from the group consisting of halogen atoms (chlorine, bromine, iodine), tosylsulfonate, trifluoromethanesulfonate, tolylsulfonatetriflate, p-bromobenzenesulfonate, 4-nitrobenzenesulfonate, tosylate, trialkyl ammonium, nitro and azide. Most preferably the leaving group is tosylsulfonate.

In a preferred embodiment of the present invention, the substitution reaction is carried out in a solvent. The solvent is preferably a aqueous solvent in order to prevent the protonation of the fluoride anion. Aprotic dipolar or aprotic polar solvents are preferred as these solvent allow more easily for the solution of ionic atoms and molecules, especially the fluoride anion. Examples of solvents which may be used in the present application include dimethylformamide (DMF), tetrahydrofurane, (THF), dimethylsulfoxide (DMSO), acetonitrile, acetamide and dimethylacetamide (DMAA).

Thus, the present invention also relates to a method, as described above, wherein the fluorination is performed in a solvent, preferably an aprotic dipolar solvent.

The reaction according to the present invention can be assisted by heating, preferably by microwave heating. Preferable combinations of solvent, heating temperature leaving group have been surprisingly found to allow for the preparation of ¹⁸F-labeled compounds by a direct fluorination method according to the present invention.

Thus, the present invention also relates to a method, as described above, wherein the fluorination is performed under microwave heating.

Preferably the fluorination is performed at a temperature in the range of 25-200° C., preferably 25-175° C., more preferably 50-150° C.

In one aspect, the present invention thus also provides a method for fluorinating derivative. This fluorination can be applied to ¹⁸F-fluoride or any other fluoride isotope. The reaction itself does not differentiate between different isotopes. As fluoride is always present within any solvent, the concentration of the ¹⁸F-fluoride of interest is measured by its radioactivity. When expressing the ratio of the radioactive fluoride in relation to the precursor for radiolabeling, the ratio of the ¹⁸F-fluoride to the precursor is in the range of 1:1,000-100,000, preferably in the range of 1:1,000-10,000 and more preferably in the range of 1:2,000-5,000.

Yields of greater than 40%, e.g. greater than 50% can also be achieved. The reactions described herein are preferably effected with no carrier added ¹⁸F.

The [¹⁸F]fluoride is present in the reaction as an anion. In order to bring this [¹⁸F]fluoride into a solution, a corresponding cation also has to be present. This cation is preferably a phase transfer catalyst. Any known phase transfer catalyst can be used. Preferred cationic phase transfer catalysts are tetraalkyl-ammonium salts.

In an embodiment of the present application, a tetrabutyl-ammonium [¹⁸F]fluoride can be used. Other ammonium salts include tetramethyl-ammonium [¹⁸F]fluoride, tetraethyl-ammonium floride, tetra(n-propyl)-ammonium floride and tetra(iso-propyl)-ammonium ¹⁸F-fluoride. The tetrabutyl ammonium [¹⁸F]fluoride may be any tetrabutyl-ammonium [¹⁸F]fluoride, i.e. the butyl may be n-butyl, iso-butyl or tert.-butyl. It will be obvious to a person skilled in the art that any tetraalkyl-ammonium salt may be used. This encompasses also ammonium salts wherein different alkyl radicals are present, e.g. dimethyl-diethyl ammonium [¹⁸F]fluoride. However, other tetraalkyl-ammonium salts may also be used, including salts wherein the alkyl groups are linear, branched or cyclic alkyl radicals. The above given definition of alkyl may also apply to the alkyl radicals of the tetraalkyl-ammonium salts.

Other feasible kationic phase transfer include crown ethers and cryptants. Preferably, a cryptand known as cryptand [2.2.2]. The most preferred cryptates are K[2.2.2]⁺ and Cs [2.2.2]⁺.

The solution containing the [¹⁸F]fluoride may preferably contain further anions to stabilize the solution. By stabilization, a constant pH value is meant. The pH of the solution should be kept in the basic region in order to prevent the [¹⁸F]fluoride from being protonated. The protonated form HF of the [¹⁸F]fluoride can not be used for the method according to the present application. To prevent this protonation, the pH of the solution should be higher than 7. This can be achieved by e.g. addition of basic anions. In a preferred embodiment, the solution of the [¹⁸F]fluoride according to the present invention contains carbonate anions. Another preferred anionic pH stabilizing anion is oxalate. Several pH stabilizing anions may also be used at the same time.

The present invention further describes use of a tracer, as described above, for the preparation of a diagnostic compound for the imaging and quantification of amyloidosis in Alzheimer's disease, type II diabetes, Down's syndrome, Creutzfeldt-Jacob disease, prion mediated diseases, amyloid polyneuropathy and amyloid cardiomyopathythe ¹⁸F-labled derivatives for use in PET diagnostics.

Still present invention provides a diagnostic method for the diagnosis of amyloid related diseases using the ¹⁸F-labled derivatives of the invention.

The objects of the present invention are achieved by the independent claims. Preferred embodiments are set forth in the dependent claims and in the examples.

Besides the above described embodiments, the present invention also describes the following alternative embodiment:

This alternative embodiment describes tracer compounds targeting aggregates of amyloid peptides in vitro or in vivo. The compounds and tracers of the invention can comprise the structure or formula:

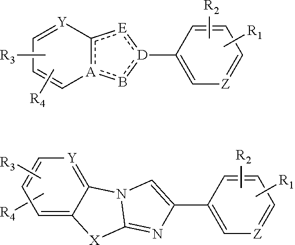

in which R₁, R₂, R₃ and R₄ are the same or different and can independently be H, F, Cl, Br, I, CN, CF₃, alkyl, alkynyl, alkoxy, monoalkylamine, dialkylamine, hydroxyalkyl, haloalkyl, alkylthio, alkylsulfonyl, aryl, heterocycles, heteroaryl, carboxy, esterified carboxy, OR₆, NR₅, R₆ or R₅, R₆ can be CₙH₂ₙ+1 or —CH₂—CH═CH-halo in which halo can be any halogen and R₆ can be CₙH₂ₙ+1, —[CH₂—CH₂—O]ₘ—R₅ where n and m can independently be an integer in the interval 0-7, A and D can independently be N or C. E, Y and Z can independently be CH or N. B can be S, O N or CH. X can be N, S, or O, and the tracers of invention can bind to and detect aggregates of amyloid peptides. In a preferred aspect, tracers of the invention can be constituted of a radionuclide including for example a photon emitting or a positron emitting radionuclide. For example a label can replace any substituent of a compound of the invention or be an additional substituent in a tracer. In one aspect, a tracer of the invention can comprise one or more of ³H, ¹¹C, ¹⁸F, ¹²³I, ¹²⁴I ⁷⁵Br, ⁷⁶Br.

According to said alternative embodiment, R¹ and R² are, independently of one another, substituted or unsubstituted, linear, branched or cyclic alkyl, alkenyl or alkynyl, optionally interrupted by one or more heteroatoms, preferably substituted or unsubstituted, linear or branched C₁-C₈ alkyl, C₂-C₈ alkenyl and C₂-C₈ alkynyl, optionally interrupted by one or more heteroatoms, more preferably substituted or unsubstituted, linear or branched C₁-C₆ alkyl, C₂-C₆ alkenyl and C₂-C₆ alkynyl, optionally interrupted by one or more heteroatoms, and most preferably substituted or unsubstituted, linear or branched C₁-C₄ alkyl, C₂-C₄ alkenyl and C₂-C₄ alkynyl, optionally interrupted by one or more heteroatoms, or halogen. R¹ and R² may also be joined together to form a ring structure containing the nitrogen atom. The most preferred ring size contains 5 or 6 ring atoms. The most preferred substituents for R₁ and R₂ are H, methyl or ethyl or a cyclic, five-membered ring, or the halogens iod or brom. Examples of compounds are given in following:

TABLE 2

| | |
|---|---|
| 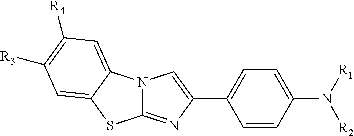 | R₁ = H, CₙH₂ₙ, CₙH₂ₙ₊₁, R₂ = H, CₙH₂ₙ, CₙH₂ₙ₊₁ R₄ = H, CₙH₂ₙ₊₁, F, Br, I or OR₅ R₃ = H, CₙH₂ₙ₊₁, F, Br, I or OR₅ where R₅ = H, CₙH₂ₙ₊₁, CₙH₂ₙF, and n is an integer from 1-6 |
| 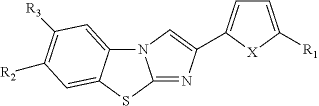 | X = N, S, O R₁ = F, Br, I, Cl R₂ = H, CₙH₂ₙ₊₁, F, Br, I OR₄ R₃ = H, CₙH₂ₙ₊₁, F, Br, I or OR₄ where R₄ = H, CₙH₂ₙ₊₁ CₙH₂ₙF and n is an integer from 1-6 |
| 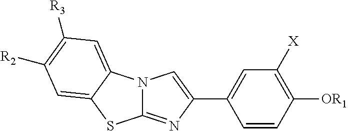 | X = F, Cl, Br, I R₁ = H, CₙH₂ₙ₊₁, CₙH₂ₙF R₂ = H, CₙH₂ₙ₊₁, F, Br, I OR₄ R₃ = H, CₙH₂ₙ₊₁, F, Br, I or OR₄ where R₄ = H, CₙH₂ₙ₊₁ CₙH₂ₙF and n is an integer from 1-6 |
| 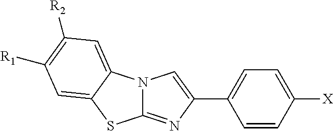 | X = F, Cl, Br, I R₁ = H, CₙH₂ₙ₊₁, F, Br, I OR₃ R₂ = H, CₙH₂ₙ₊₁, F, Br, I or OR₃ where R₃ = H, CₙH₂ₙ₊₁ CₙH₂ₙF and n is an integer from 1-6 |
| 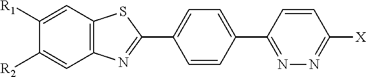 | X =F, Cl, Br, I R1= H, CnH2n + 1, F, Br, I OR3 R2 = H, CnH2n + 1, F, Br, I or OR3 where R3 = H, CnH2n + 1 CnH2nF and n is an integer from 1-6 |
| 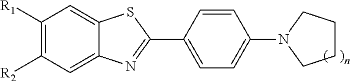 | R1 = H, CnH2n + 1, F, Br, I or OR3 R2 = H, CnH2n + 1, F, Br, I or OR3 where R3 = H, CnH2n +1, CnH2nF, and n is an integer from 1-3 |
| 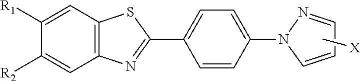 | X = F, Br, I R1 = H, CnH2n + 1, F, Br, I or OR3 R2 = H, CnH2n + 1, F, Br, I or OR3 where R3 = H, CnH2n + 1, CnH2nF, and n is an integer from 1-6 |
| 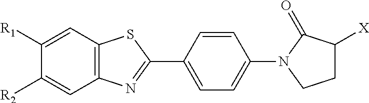 | X = F, Cl, Br, I R1 = H, CnH2n + 1, F, Br, I or OR3 R2 = H, CnH2n + 1, F, Br, I or OR3 where R3 = H, CnH2n + 1, CnH2nF, and n is an integer from 1-6 |
| 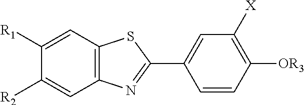 | X = H, F, Cl, Br, NH2, O, OR3 R1 = H, CnH2n + 1, F, Br, I or OR3 R2 = H, CnH2n + 1, F, Br, I or OR3 where R3 = H, CnH2n + 1, CnH2nF, and n is an integer from 1-6 |
| 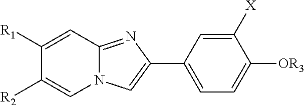 | X = H, F, Cl, Br, NH2, O, OR3 R1 = H, CnH2n + 1, F, Br, I or OR3 R2 = H, CnH2n + 1, F, Br, I or OR3 where R3 = H, CnH2n + 1, CnH2nF, and n is an integer from 1-6 |
| 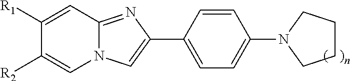 | R1 = H, CnH2n + 1, F, Br, I or OR3 R2 = H, CnH2n + 1, F, Br, I or OR3 where R3 = H, CnH2n + 1, CnH2nF, and n is an integer from 1-3 |

Further, the invention also describes, according to said alternative embodiment, the preparation of these compounds comprising the structure

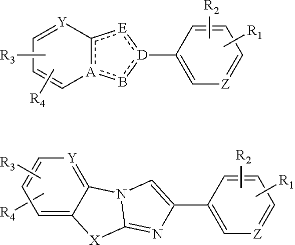

where one or more of R₁, R₂, R₃, R₄ contains a fluor atom bound to an alkyl chain. The term alkyl as used within this application includes any linear, branched or cyclic saturated hydrocarbon. Preferably, the alkyl is a C₁ to C₁₀ alkyl, more preferably a C₁ to C₈ alkyl and even more preferably a C₁ to C₆ alkyl. However, this carbon chain may optionally be substituted or interrupted by heteroatoms like N, O and S. Preferred are interruptions by up to four heteroatoms within the carbon chain. Examples of these interruptions include ethers, thioethers, amines and polyethylene glycols (PEGs). Preferred aliphatic chains with heteroatom interruptions include PEG with 2 to 6 PEG groups and their corresponding polythioether equivalents. Further, the PEG substituents contain a halogen substitution, preferably a terminal halogen substitution.

Optionally, the alkyl may further be substituted. Substitution include halogen atoms, groups containing heteroatoms like OR, NR₂, SR, SCN, CN, COOR, wherein R is H, alkyl, alkenyl, alkynyl or aryl.

The term “alkenyl” as used in the context of said alternative embodiment refers to an unsaturated straight of branched chain hydrocarbon radical comprising at least one carbon-to-carbon double bond. The chain can be constituted of up to 8 carbons, preferably 5 carbons, more preferably 4 carbons. Examples include, but are not limited to ethenyl, propenyl, iso-propenyl, butenyl, iso-butenyl, t-butenyl. Furthermore, “alkenyl” can refer to an unsaturated straight or branched chain hydrocarbon radical comprising at least one carbon-to-carbon triple bound. The chain can be constituted of up to 8 carbons, preferably 5 carbons, more preferably 4 carbons. Examples include, but are not limited to ethylyl, propynyl, iso-propynyl, butynyl, isobutynyl, t-butynyl. A compound or tracer of the invention can comprise one or more alkenyl or alkynyl substituents.

The term “alkoxy” as used in the context of said alternative embodiment refers to a straight or branched chain alkyl radical bonded to an oxygen atom of a chain length of 1-8 carbons, preferably 1-6 carbons, more preferably 1-4 carbons. Examples include, but are not limited to, methoxy, ethxoy, n-propoxy, isopropoxy, butoxy, iso-butoxy, t-butoxy. A compound or tracer of the invention can comprise one or more alkoxy substituents.

The term “monoalkylamine” by itself or as a part of another group as used in the context of said alternative embodiment refers to an amino group that is substituted with one alkyl group. In one aspect, the term “methylamino” can refer to a neutral group or ring substituent in which N is connected to a compound of the invention via the ring or a chain of the compound and N is further bound to a methyl and a hydrogen. The N may be charged and form a salt. A compound or tracer of the invention can comprise one or more monoalkylamine substituents.

The term “dialkylamine” by itself or as a part of another group as used in the context of said alternative embodiment refers to an amino group that is substituted with two alkyl groups. In one aspect, the term “dimethylamino” can refer to a neutral group or ring substituent in which N is connected to a compound of the invention via the ring or a chain of the compound and N is further bound to two methyl groups. The N may be charged and form a salt. A compound or tracer of the invention can comprise one or more dialkylamine substituents.

The term “hydroxy(C₁₋₅)alkyl” as used in the context of said alternative embodiment refers to an alkyl chain connected to a compound of the invention via the ring or chain of the compound in which the distal portion of the alkyl chain of the group contains a hydroxy moiety. Preferably, the number of carbons in the alkyl chain is from 1 to 5. A compound or tracer of the invention can comprise one or more hydroxy(C₁₋₅)alkyl substituents.

The term “halo” or “halogen” by itself or as a part of another group as used in the context of said alternative embodiment refers to fluorine, chlorine, bromine or iodine. A compound or tracer of the invention can comprise one or more halo substituents.

The term “haloalkyl” as used in the context of said alternative embodiment refers to any of the mentioned alkyl groups substituted by one or more fluorine, chlorine, bromine or iodine such as chloromethyl, iodomethyl, trifluoromethyl, 2,2,2-trifluoroethyl and 2-chloroethyl. A compound or tracer of the invention can comprise one or more haloalkylsubstituents.

The term “alkylthio” by itself or as a part of another group as used in the context of said alternative embodiment refers to a thioether of the structure R*-S in which R* is a C₁₋₄ alkyl. A compound or tracer of the invention can comprise one or more alkylthio substituents.

The term “heterocycle” or “heterocyclic ring” as used in the context of said alternative embodiment represent a 4 to 7-membered mono-heterocyclic ring that may be saturated or unsaturated, and consist of carbon atoms and from one to three heteroatoms selected from the group consisting of N, O and S. Examples include, but are not limited to piperidinyl, pyrrolyl, pyrrolidinyl, imidazolyl, imidazinyl, imidazolinyl, pyridyl, pyrazinyl, pyriminidyl, oxaxzolyl, oxazolidinyl, isoxazolyl; isoxazolindinyl, thiazolyl, thiazolidinyl, isothiazolyl, homopiperidinyl, homopiperazinyl, pyridazinyl, pyrazolyl, pyrazolidinyl. A compound or tracer of the invention can comprise one or more heterocycle or heterocyclic ring substituents.

The term “heteroatom” as used in the context of said alternative embodiment represent an oxygen atom (“O”), a sulphur atom (“S”) or a nitrogen atom (“N”). It will be recognized that when the heteroatom is nitrogen, it may form an NR′R″ in which R′ and R″ are, independent from another, hydrogen, C₁₋₄ alkyl, C₂₋₄ aminoalkyl, C₁₋₄ haloalkyl or halo benzyl. R′ and R″ can be taken together to form a 5 to 7-member heterocyclic ring that optionally comprises O, S or NR′″ in which R′″ is hydrogen or C₁₋₄ alkyl. A compound or tracer of the invention can comprise one or more heteroatom substituents.

The following alternative embodiments are described:

1. A tracer for aggregates of amyloid peptides comprising the structure

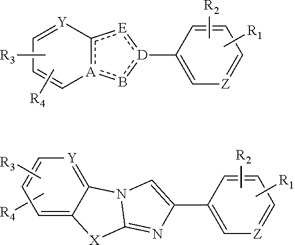

in which R₁, R₂, R₃ and R₄ are the same or different and can independently be H, F, Cl, Br, I, CN, CF₃, alkyl, alkynyl, alkoxy, monoalkylamine, dialkylamine, hydroxyalkyl, haloalkyl, alkylthio, alkylsulfonyl, aryl, heterocycles, heteroaryl, carboxy, esterified carboxy, OR_{B}, NR₅, R₆ or R₅, R₆ can be CₙH₂ₙ+1 or —CH₂—CH═CH-halo in which halo can be any halogen and R₆ can be CₙH₂ₙ+1, —[CH₂—CH₂—O]ₘ—R₅ where n and m can independently be an integer in the interval 0-7, A and D can independently be N or C. E, Y and Z can independently be CH or N. B can be S, O N or CH. X can be N, S, or O, and the tracers of invention can bind to and detect aggregates of amyloid peptides. Preferably, the alkyl is a C₁ to C₁₀ alkyl, more preferably a C₁ to C₈ alkyl and even more preferably a C₁ to C₆ alkyl. However, this carbon chain may optionally be substituted or interrupted by heteroatoms like N, O and S. Preferred are interruptions by up to four heteroatoms within the carbon chain. Examples of these interruptions include ethers, thioethers, amines and polyethylene glycols (PEGs). Preferred aliphatic chains with heteroatom interruptions include PEG with 2 to 6 PEG groups and their corresponding polythioether equivalents. In another preferred embodiment, the PEG substituents contain a halogen substitution, preferably a terminal halogen substitution.
2. A tracer for aggregates of amyloid peptides of embodiment 1, wherein the compound comprises the formula:

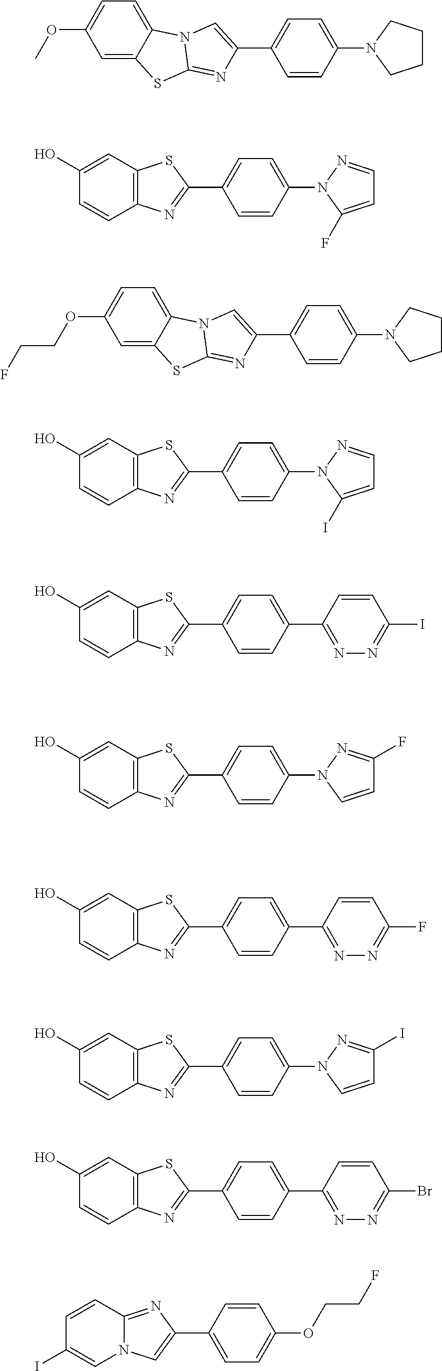

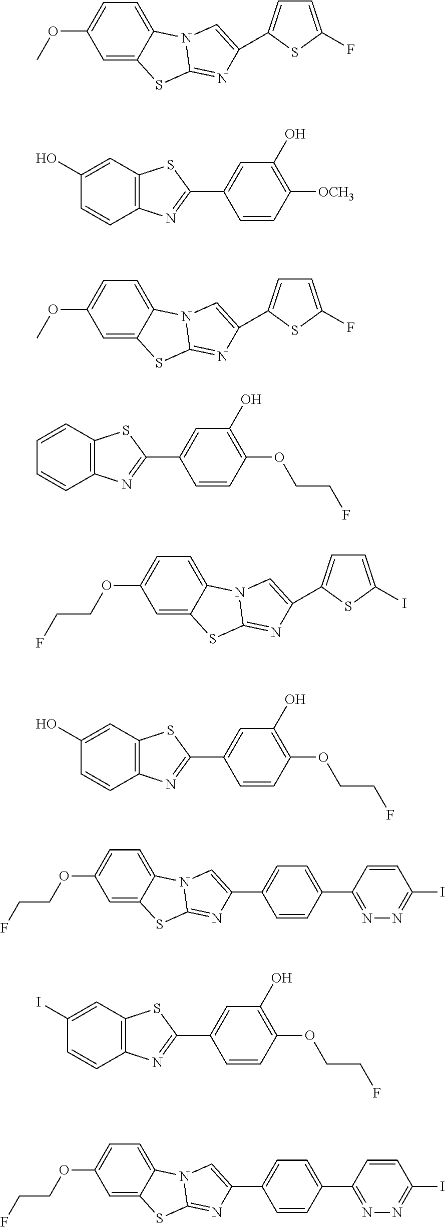

3. A method for the preparation of a tracer of embodiment 1 comprising the general formula II

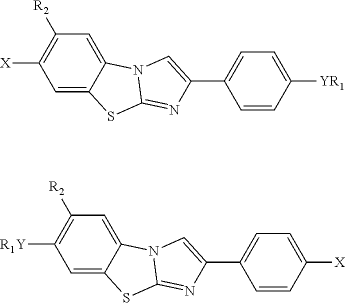

wherein

### X═F, Br, I

### Y═O, N, S.

R₁═H, alkyl, alkenyl, haloalkyl, hydroxyalkyl, polymers of ethylene glycol
R₂═H, alkyl, alkenyl, haloalkyl, hydroxyalkyl
by fluorinating a derivative of the general formula I

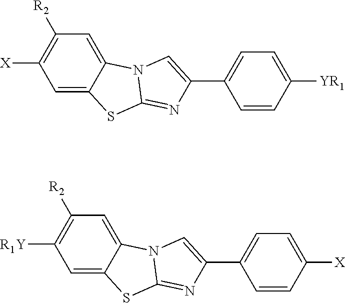

wherein

### X═F, Br, I

### Y═O, N, S.

R₁═H, alkyl, alkenyl, haloalkyl, hydroxyalkyl, polymers of ethylene glycol each containing a leaving group.
R₂═H, alkyl, alkenyl, haloalkyl, hydroxyalkyl each containing a leaving group.
with [¹⁸F]fluoride.
   4. The method according to embodiment 3, wherein the leaving group is selected from the group consisting of halogen atoms (chlorine, bromine, iodine), tosylsulfonate, trifluoromethanesulfonate, tolylsulfonatetriflate, tosylate, trialkyl ammonium, nitro and azide.
   5. The method according to any of the preceding embodiment 3, wherein the fluorination is performed in a solvent, preferably an aprotic dipolar solvent.
   6. The method according to embodiment 5, wherein the solvent is selected from the group consisting of dimethylformamide (DMF), tetrahydrofurane, (THF), dimethylsulfoxide (DMSO), acetonitrile and acetamide
   7. The method according to any of the preceding embodiments, wherein the fluorination is performed under microwave heating.
   8. The method according to any of the preceding embodiments, wherein the fluorination is performed at a temperature in the range of 0.25-200° C., preferably 25-175° C., more preferably 50-150° C.
   9. The method according to any of the preceding embodiments [¹⁸F]fluoride to the precursor derivative is in the range of 1:1,000-100,000, preferably in the range of 1:1,000-10,000 and more preferably in the range of 1:2,000-5,000.
   10. The method according to any of the preceding embodiments, wherein a compound of one of the general formulas is comprising the following formulas:

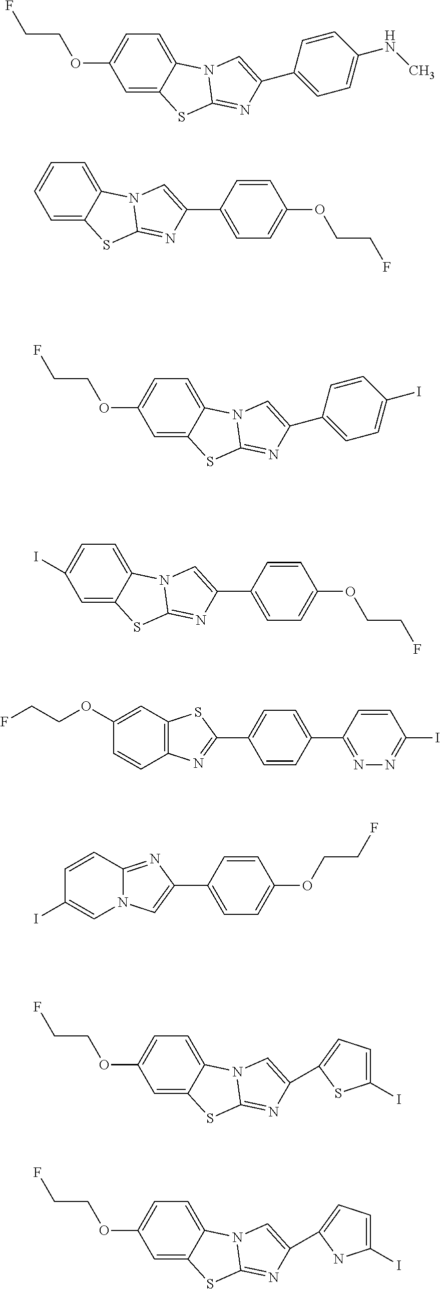

11. A tracer for aggregates of amyloid peptides of embodiment 1 wherein at least one of R₁, R₂, R₃ and R₄ independently comprises ¹¹C, ¹⁸F, ¹⁹F, ⁷⁵Br, 76Br, ¹²³I, ¹³¹I, ¹²⁴I
12. Use of a tracer according to embodiment 3 for the preparation of a diagnostic compound for the imaging and quantification of amyloidosis in Alzheimer's disease, type II diabetes, Down's syndrome, Creutzfeldt-Jacob disease, prion mediated diseases, amyloid polyneuropathy and amyloid cardiomyopathy.

The following examples are provided to exemplify the invention and are nor meant to be limiting in any way:

### Example 1

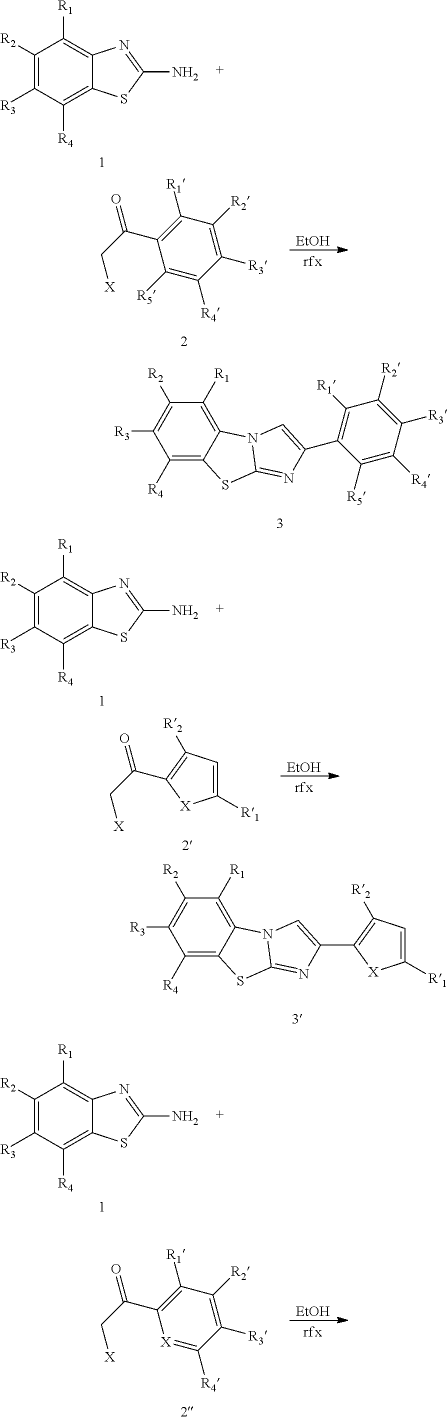

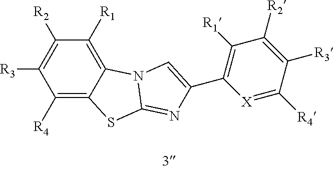

### Synthesis of a Set of Compounds

2-Phenylimidazo[2,1-b]benzothiazoles 3 were prepared from the appropriately substituted 2-aminobenzothiazoles 1 and the appropriately substituted phenacylhalides 2. Treatment of 1 with equimolar amount of 2 in ethanol at refluxing temperature afforded 3 in moderate to excellent yields (37-95%). All the products and intermediates were characterized by mass spectrometry and their purities checked by HPLC. Selected compounds were also confirmed by ¹H-NMR and ¹³C-NMR.

Similarly, 2-heteroaryl imidazo[2,1-b]benzothiazoles were prepared from the appropriately substituted 2-aminobenzothiazoles 1 and the appropriately substituted 2-bromo-1-(heteroaryl)ethanones (2′ or 2″).

### Example 1.1 (Comparative)

### 4-(7-Methoxyimidazo[2,1-b]benzothiazol-2-yl)-N,N-diethyl-benzenamine (1)

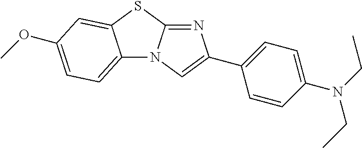

A mixture of 6-methoxybenzo[d]thiazol-2-amine (180 mg, 1 mmol) and 2-bromo-1-(4-(diethylamino)phenyl)ethanone (270 mg, 1 mmol) in 5 ml EtOH was heated overnight at reflux. The reaction mixture was cooled to room temperature, filtered, washed with 2 ml ether and the precipitate, 4-(7-methoxyimidazo[2,1-b]benzothiazol-2-yl)-N,N-diethyl-benzenamine, was dried under vacuum (263 mg, 75% yield, >95% as measured by HPLC purity). ESI-MS [M+1]=352.1; DMSO-d₆ ¹H NMR δ 1.07 (6H, t), 3.63 (4H, q), 3.83 (3H,s), 7.18 (2H, d), 7.72 (2H,s), 7.93 (2H, d), 8.80 (1H,s), 11.22 (1H,s); ¹³C NMR 11.3, 56.8, 100.0, 102.2, 108.2, 114.9, 115.0, 126.6, 126.9, 131.4, 148.9, 158.0.

### Example 2 (Comparative)

### 4-(7-Hydroxyimidazo[2,1-b]benzothiazol-2-yl)-N,N-diethyl-benzenamine (2)

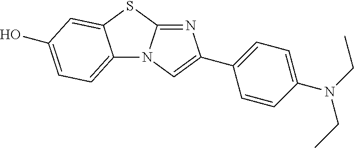

Compound 1 (105 mg, 0.3 mmol) was reacted with 1.5 molar equivalents of BBr₃ (1 molar solution) in 10 ml dichloromethane under microwave heating at 150° C. for 20 min. The reaction mixture was quenched with 10 ml water and extracted with 2×10 ml CH₂Cl₂. The combined organic phase was washed with saturated Sodium bicarbonate solution (10 ml), dried over sodium sulphate and evaporated. The crude product, 2, 4-(7-hydroxyimidazo[2,1-b]benzothiazol-2-yl)-N,N-diethyl-benzenamine, was recrystallized from MeOH (92 mg, 91% yield) and used for further ¹¹C-methylation reaction. ESI-MS, [M+1]=338.1; DMSO-d₆ ¹H NMR δ1.10 (6H, t), 3.35 (4H, q), 6.69 (2H, d), 6.92 (1H, d), 7.35 (1H,s), 7.62 (2H, d), 7.73 (1H, d), 8.35 (1H,s), 9.83 (1H,s); ¹³C NMR δ 11.4, 44.5, 107.1, 111.6, 112.4, 114.4, 115.1, 122.1, 125.9, 126.7, 131.0, 146.2, 147.5, 147.6, 155.7.

As an alternative procedure, 2 was also prepared from 1 by using 3 equivalent of 1 molar solution of BBr₃ in dichloromethane at room temperature during 48 h.

### Example 3 (Comparative)

### 4-(7-Methoxyimidazo[2,1-b]benzothiazol-2-yl)-N,N-dimethyl-benzenamine (3)

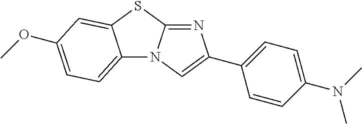

A mixture of 6-methoxybenzo[d]thiazol-2-amine (180 mg, 1 mmol) and 2-bromo-1-(4-(dimethylamino)phenyl)ethanone (242 mg, 1 mmol) in 5 ml EtOH was heated overnight at reflux. The reaction mixture was cooled to room temperature, filtered, and washed with 2 ml ether and the resulting precipitate, 4-(7-methoxyimidazo[2,1-b]benzothiazol-2-yl)-N,N-dimethyl-benzenamine, dried under vacuum (246 mg, 76% yield, purity>95% as measured by HPLC ESI-MS, [M+1]=324.1; DMSO-d₆ ¹H NMR δ 3.06 (6H,s), 3.85 (3H,s), 6.55 (2H, d), 7.38 (2H, d), 7.72 (3H, m), 7.98 (2H, d), 8.80 (1H,s); ¹³C NMR δ 43.0, 56.8, 108.3, 109.4, 110.4, 111.6, 113.0, 115.3, 126.5, 126.7, 131.4, 146.1, 158.3.

### Example 4 (Comparative)

### 4-(7-hydroxyimidazo[2,1-b]benzothiazol-2-yl)-N,N-dimethyl-benzenamine (4)

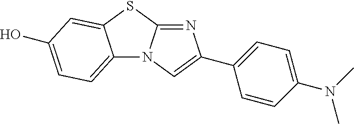

Compound 3 (97 mg, 0.3 mmol) was reacted with 1.5 molar equivalents of BBr₃ (1 molar solution) in 10 ml dichloromethane under microwave heating at 150° C. for 20 min. The reaction mixture was quenched with 10 ml water and extracted with 2×10 ml CH₂Cl₂. The combined organic phase was washed with saturated Sodium bicarbonate solution (10 ml), dried over sodium sulphate and evaporated. The crude product, 4-(7-hydroxyimidazo[2,1b]benzothiazol-2-yl)-N,N-dimethyl-benzenamine, 4, was recrystallized from MeOH (87 mg, 90% yield) and used further for ¹¹C-methylation reaction. LC-MS-ESI, [M+1]=310.1; DMSO-d₆¹H NMR δ 2.89 (6H,s), 5.88 (1H,s), 6.26 (1H,s) 6.77 (2H, d), 7.65 (2H, d), 7.88 (1H, m), 8.41 (1H, s), 9.85 (1H, s); ¹³C NMR δ 38.0, 107.4, 111.6, 113.3, 114.5, 114.9, 125.5, 128.6, 129.3, 130.2, 133.3, 134.6, 136.8, 147.3, 155.8.

### Example 5

### 2-(4-(pyrrolidin-1-yl)phenyl)-7-methyoxyimidazo[2,1-b]benzothiazole (5)

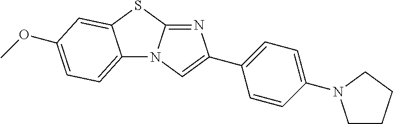

The title compound was prepared in a similar manner as given in Example 1 by using 1 mmol of 6-methoxybenzo[d]thiazol-2-amine and 2-bromo-1-(4-(pyrrolidin-1-yl)phenyl)ethanone to afford (295 mg, 84% yield in a purity>95% as measured by HPLC). ESI-MS, [M+1]=350.2; DMSO-d₆ ¹H NMR δ 1.97 (4H, t), 3.28 (4H, t), 3.85 (3H,s), 6.66 (2H, d), 7.28 (1H, d), 7.61 (2H, d), 7.83 (1H,s), 8.05 (1H, d), 8.78 (1H,s); ¹³C NMR δ 25.8, 48.4, 56.8, 108.3, 110.4, 112.6, 115.8, 126.4, 126.9, 127.5, 131.0, 131.5, 141.9, 145.9, 148.5, 158.6.

### Example 6

### 2-(4-(Pyrrolidin-1-yl)phenyl)-7-hydroxyimidazo[2,1-b]benzothiazole (6)

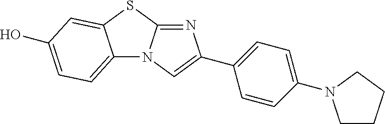

Compound 5 2-(4-(Pyrrolidin-1-yl)phenyl)-7-methyoxyimidazo[2,1-b]benzothiazole, (105 mg, 0.3 mmol) was reacted with 1.5 molar equivalents of BBr₃ (1 molar solution) in 10 ml dichloromethane and microwave irradiation at 150° C. for 20 min. The reaction mixture was quenched by adding water and extracted with 2×10 ml CH₂Cl₂. The combined organic phase was washed with saturated Sodium bicarbonate solution (10 ml), dried over sodium sulphate and evaporated. The crude product, 2-(4-(pyrrolidin-1-yl)phenyl)-7-hydroxyimidazo[2,1-b]benzothiazole, was recrystallized from MeOH (89 mg, 88% yield) and further used for ¹¹C-methylation reaction. ESI-MS, [M+1]=336.1; DMSO-d₈ ¹H NMR δ 1.86 (4H,s), 3.14 (4H,s), 5.32 (OH, br), 6.46 (2H, d), 7.12 (1H, d), 7.39 (2H,d), 7.61 (1H,s), 8.00 (1H, d), 8.84 (1H,s); ¹³C NMR δ 25.7, 48.4, 108.4, 111.9, 116.0, 116.7, 125.1, 127.0, 127.6, 131.4, 131.7, 140.1, 145.4, 148.0, 156.2.

### Example 7

### 4-(7-Methoxyimidazo[2,1-b]benzothiazol-2-yl)-benzenamine (7)

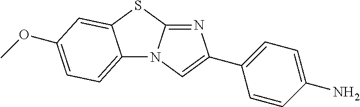

A mixture of 6-methoxybenzo[d]thiazol-2-amine (180 mg, 1 mmol) and 2-bromo-1-(4-nitrophenyl)ethanone (244 mg, 1 mmol) in 5 ml EtOH was heated for 1 h at reflux. The reaction mixture was cooled to room temperature, filtered, washed with 2 ml ether and the precipitate, 4-(7-methoxyimidazo[2,1-b]benzothiazol-2-yl)-nitrobenzene dried under vacuum (321 mg, 99% yield with HPLC purity of >95%). Thereafter, a mixture of the nitro derivative (321 mg, 0.99 mmol) and SnCl₂ (5 eq., 690 mg) in 20 ml EtOH was heated for 2 h at reflux temperature under nitrogen. The reaction mixture was concentrated under vacuum and the residue taken to 200 ml ethyl acetate, the organic phase washed with 1M NaOH solution, followed by water, and was subsequently dried over sodium sulphate and the solution concentrated. The crude product, 4-(7-methoxyimidazo[2,1-b]benzothiazol-2-yl)-benzenamine 7 was recrystallized from MeOH, dried under high vacuum and directly used as precursor for radiosynthesis of [¹¹C]8. LC-MS-ESI, [M+1]=296.1

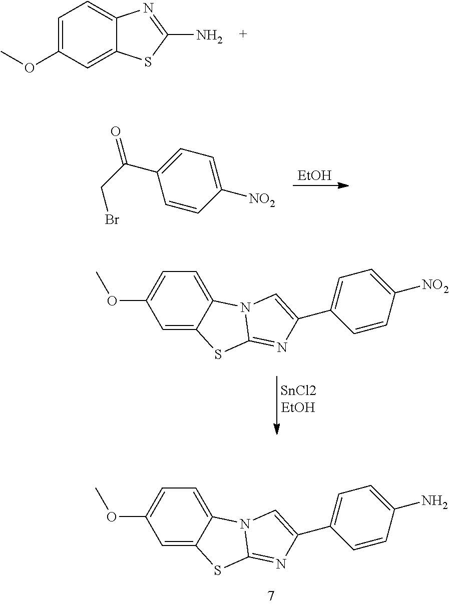

### Synthesis of 7

### Example 8

### 2-(p-Methylaminophenyl)-7-methoxyimidazo[2,1-b]benzothiazole (8)

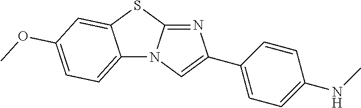

Method 1: Compound 7 (295 mg, 1 mmol) was dissolved in DMF (10 ml) and treated with potassium carbonate (anhydrous, 2 eq., 280 mg). One equivalent of methyl iodide was added to the mixture at 140° C. The reaction mixture was cooled to room temperature, 50 ml water was added, and the resulting mixture was extracted with dichloromethane (3×50 ml). The combined organic phase was washed with brine, dried over sodium sulphate and concentrated. The crude product was purified by flash chromatography. ESI-MS, [M+1]=310.1; ¹H NMR (DMSO-d₆) δ 2.7 (d, J=5 Hz, 3H), 3.3 (s, 3H), 5.7 (br s, 1H), 6.6 (d, J=8.5 Hz, 2H), 7.1 (q, J_{A}=8.5, Jₛ=2.5 Hz, 1H), 7.6 (m, 4H), 8.4 (s, 1H); ¹³C NMR (DMSO-d₆) δ 30.2, 56.3, 106.8, 109.9, 112.1, 114.1, 118.8, 122.2, 126.1, 126.6, 130.7, 146.1, 147.6, 149.7, 157.1.

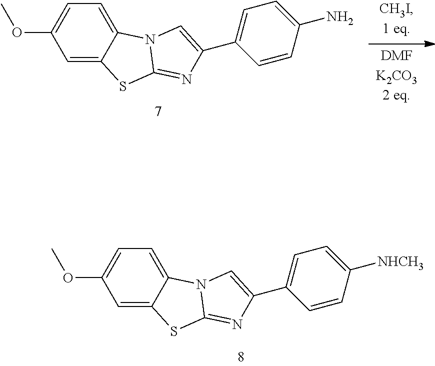

### Synthesis of 8

Method 2: Alternatively, 8 was prepared by reacting 6-methoxybenzo[d]thiazol-2-amine (180 mg, 1 mmol) with N-(4-(2-bromoacetyl)phenyl)acetamide (256 mg, 1 mmol) in 5 ml EtOH for 2 h at reflux. The reaction mixture was cooled to room temperature, filtered, washed with 2 ml ether and the precipitate, N-acetyl-4-(7-methoxyimidazo[2,1-b]benzothiazol-2-yl)-benzenamine, 9 was dried under vacuum (318 mg, 94% yield, HPLC purity of >95%). 9 was methylated with 2 eq. methyltriflate in 30 ml acetone at 60° C. in a for 10 min in a closed vial. After this, 200 ml water was added and the solution filtered. The intermediate, 10, was treated with 2M NaOH at 100° C. for 10 min. using microwave heating. Workup afforded 8 in >95% purity (HPLC).

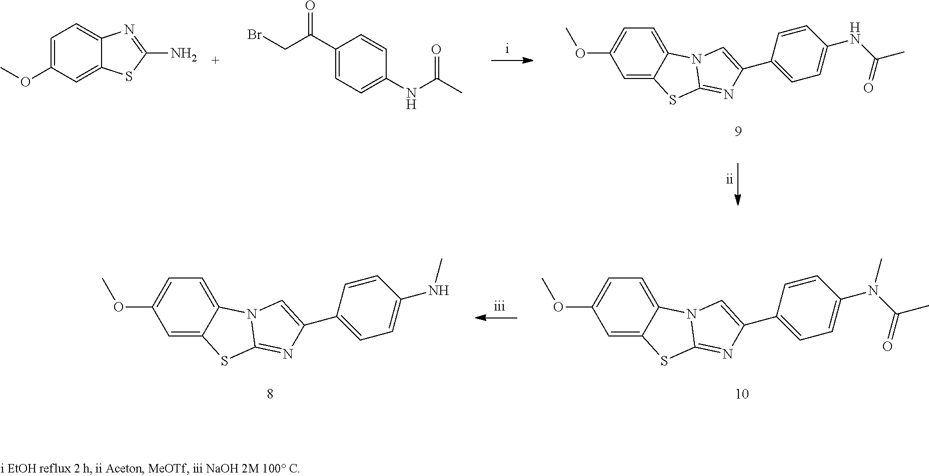

### Example 9

### 2-(m-Fluoro-p-methoxyphenyl)-7-chloroimidazo[2,1-b]benzothiazole(11)

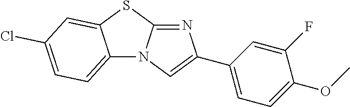

A mixture of 6-chlorobenzo[d]thiazol-2-amine (185 mg, 1 mmol) and 2-bromo-1-(3-fluoro-4-methoxyphenyl)ethanone (247 mg, 1 mmol) in 5 ml EtOH was heated overnight at reflux. The reaction mixture was cooled to room temperature, filtered, washed with 2 ml ether and the precipitate, 2-(m-fluoro-p-methoxyphenyl)-7-chloroimidazo[2,1-b]benzothiazole 11 was dried under vacuum (252 mg, 76% yield, purity of >95%.as measured by HPLC). ESI-MS, [M+1]=333.1; ¹H NMR (DMSO-d₆) δ 3.88 (s, 3H), 7.24 (t, 1H), 7.63 (m, 3H), 7.95 (d, 1H), 8.22 (d, 1H), 8.72 (s, 1H); ¹³C NMR (DMSO-d₆) δ 57.0, 109.8, 113.0, 115.1, 121.8, 125.6, 127.7, 129.9, 131.6, 131.9, 146.1, 147.3, 147.4, 147.9, 151.2, 153.9.

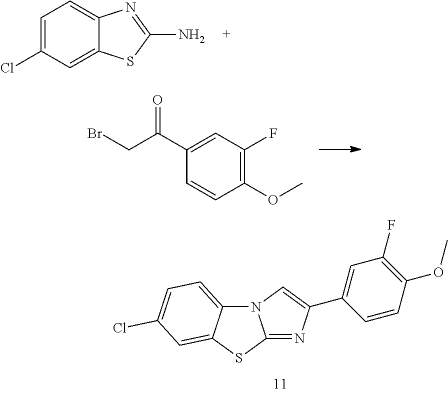

### Preparation of 11

### Example 10

### 2-(p-Methylaminophenyl)-7-(2-fluoroethoxy)imidazo[2,1-b]benzothiazole (12)

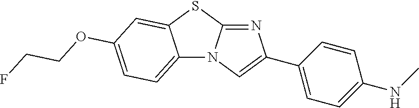

N-Acetyl-N-methyl-4-(7-methoxyimidazo[2,1-b]benzothiazol-2-yl)-benzenamine, 10 (105 mg, 0.3 mmol) was reacted with 2 equivalents of BBr₃ (1 molar solution) in 10 ml dichloromethane under microwave irradiation at 120° C. for 30 min. The reaction mixture was quenched by adding water and thereafter extracted with 2×10 ml CH₂Cl₂. The combined organic phase was washed with saturated sodium bicarbonate solution (10 ml), dried over sodium sulphate and concentrated. The crude product, N-acetyl-N-methyl-4-(7-hydroxyimidazo[2,1-b]benzothiazol-2-yl)-benzenamine 13, was deprotonated with NaH (2 eq.) in 10 ml DMF followed by reaction with 1 equivalent of 1-bromo-2-fluoroethane at 80° C. The reaction was followed by TLC and after 15 min no starting material was detected. The reaction mixture was cooled to room temperature. 100 ml water was added, and the mixture extracted with 3×50 ml ethyl acetate. The combined organic phase was washed with saturated sodium bicarbonate, brine, dried over sodium sulphate and concentrated. The product was dissolved in 5 ml EtOH and directly deprotected by treatment with 20 ml of a 2 M NaOH solution at 100° C. for 10 min. using microwave heating. The reaction mixture was quenched with water and extracted with dichloromethane 3×50 ml, washed with saturated sodium bicarbonate, brine and dried over sodium sulphate. The organic phase was evaporated to give 15 (80 mg, 78% overall yield, purity>95%). ESI-MS, [M+1]=342.1; ¹H NMR (DMSO-d₆) δ 2.71 (s, 3H), 4.32 (m, 2H, J_{HF}=32 Hz), 4.79 (m, 2H, J_{HF}=48 Hz), 5.75 (s, 1H), 6.59 (d, 12H), 7.18 (dd, 1H), 7.58 (d, 2H), 7.68 (d, 1H), 7.85 (d, 1H), 8.40 (s, 1H); ¹C NMR (DMSO-d₆) δ 30.6, 68.6, 83.8, 100.6, 107.2, 111.2, 112.6, 115.5, 115.1, 126.5, 129.6, 131.1, 139.9, 150.1, 154.3, 156.3.

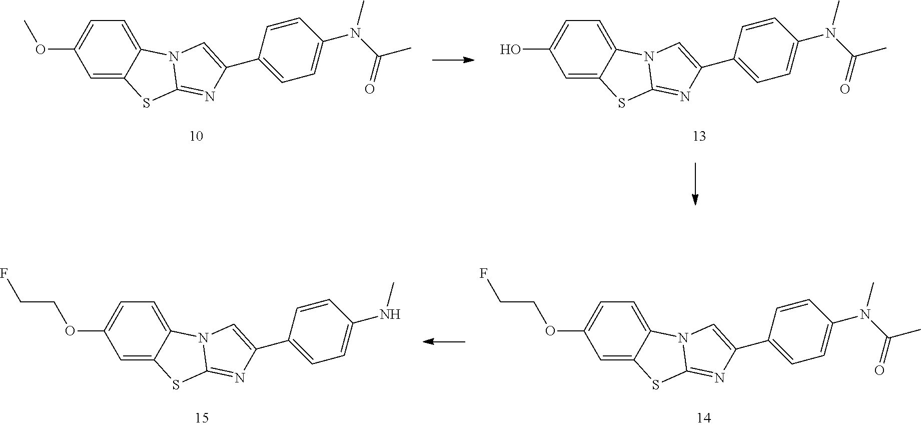

### Synthesis of 15

### Example 11

### Compounds of the Invention

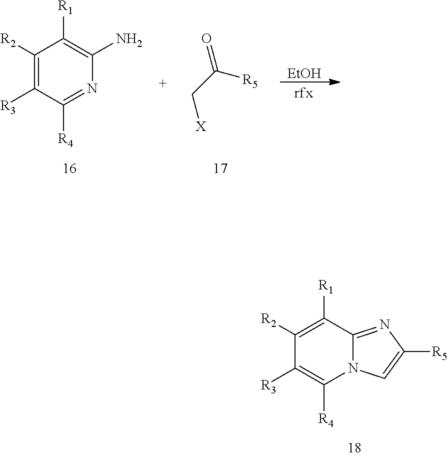

### Syntheses of imidazo[2,1-a]pyridines

Most of the new imidazo[2,1-a]pyridines 18 were synthesized through direct condensation of the appropriately substituted 2-aminopyridines 16 and the appropriately substituted α-bromoketones 17. Treatment of 16 with 1.2 equivalents of 17 in ethanol at refluxing temperature afforded 18 in 25 to 93% yield.

Whereas in formula 16 R₁-R₄ is hydrogen, nitro, halide, hydroxyl, alkoxy, halo alkoxy 1′, 2′ or 3′ amine, halo alkylamines, cyclic and acyclic groups and in formula 17, R₅ is aryl, heteroaryl, bicyclic compounds and their substituted aromatic and heteroaromatic systems wherein substitutions are, in one or more positions, nitro, halide, hydroxyl, alkoxy, halo alkoxy 1′, 2′ or 3′ amine, halo alkylamines, cyclic and acyclic groups.

### Example 12

### 6-chloro-2-(5-chlorothiophen-2-yl)imidazo[1,2-a]pyridine, (19)

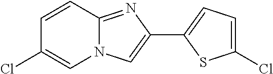

2-Amino-5-chloropyridine (128.5 mg, 1 mmol) and 2-bromo-1-(5-chlorothiophen-2-yl)ethanone (288 mg, 1.2 mmol) were stirred in reflux ethanol (5 mL) for 4 h. The mixture was allowed to cool down to room temperature while stirring and then filtered. The white solid was washed with diethyl ether and dried under vacuum to give 218 mg of product. Yield 81%; >99% HPLC purity; ESI-MS, [M+1]=269.0; DMSO-d₆ ¹H NMR δ 8.88 (1H,s), 8.35 (1H,s), 7.67 (1H, d), 7.42 (1H, d), 7.32 (1H, d), 7.18 (1H, d); ¹³C NMR δ 143.2, 129.2, 128.9, 128.4, 1126.1, 125.3, 121.0, 117.4, 110.3.

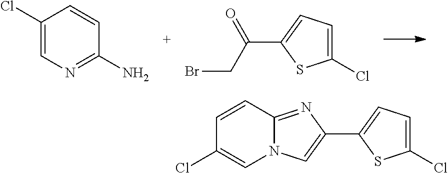

### Syntheses of 19

### Example 13

### Compounds of the Invention

The benzothiazoles 24 were prepared from 2-aminothiophenoles 21 and the appropriately substituted aldehydes 22 or acylchlorides 25 based on previously reported methods (Henriksen et al. J Med Chem 2007; 50: 1087-1089) afforded 24 in 46-92% yield. Similarly, the benzothiazoles 27 were prepared from 2-aminothiophenole 26 and the appropriately substituted aldehydes 22 or acylchlorides 25.

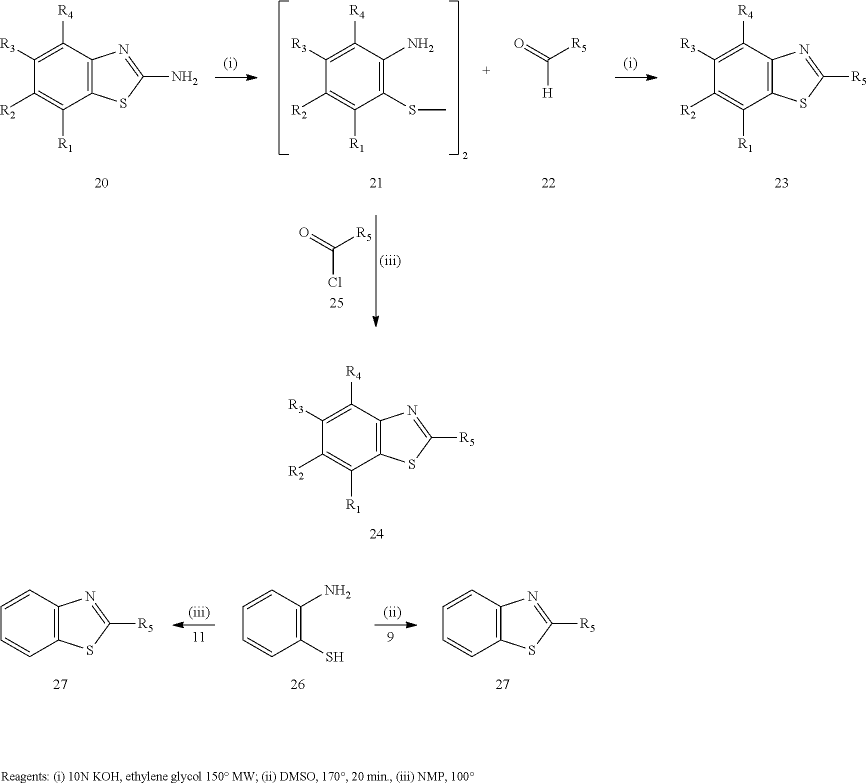

Whereas in formula 20 and 21 R₁-R₄ is hydrogen, nitro, halide, hydroxyl, alkoxy, halo alkoxy 1′, 2′ or 3′ amine, halo alkylamines, cyclic and acyclic groups and in formula 22 and 25, R₅ is aryl, heteroaryl, bicyclic compounds and their substituted aromatic and heteroaromatic systems wherein substitution in one or more positions, is nitro, halide, hydroxyl, alkoxy, halo alkoxy 1′, 2′ or 3′ amine, halo alkylamines, cyclic and acyclic groups.

### Example 14

### 2-(4-(6-chloropyridazin-3-yl)phenyl)-6-methoxybenzo[d]thiazole, 27a

The 2-amino-5-methoxythiophenol (156 mg, 0.5 mmol) and 4-(6-chloropyridazin-3-yl)-benzaldehyde (218 mg, 1 mmol) were stirred at 170° C. in DMSO (3 mL) for 20 min. The reaction mixture was cooled to room temperature and poured into ice-water. The precipitate was filtered under reduced pressure, washed with diethyl ether and recrystallized from 70% ethanol to furnish 184.6 mg (52%) of 27a as a pale-yellow solid with 98% HPLC purity; ESI-MS, [M+1]=354.1; DMSO-d₆ ¹H NMR δ 8.38 (2H,d), 8.13 (1H,d), 7.67 (2H, d), 7.42 (2H, m), 7.11 (1H, d), 7.07 (1H, d), 3.68 (3H, s).

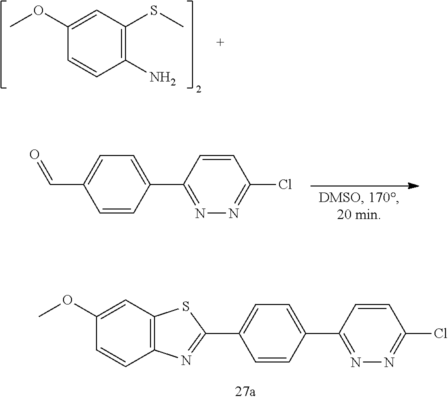

### Syntheses of 27a

### Example 15

### Determination of test compound's inhibition of N-[³H-methyl]6-OH-BTA-1 binding to fibrils of Aβ₄₀ and Aβ₄₂

Human Aβ₄₀ and Aβ₄₂ peptides (Bachem) were incubated at 0.5 mg/ml in a solution of 10 mM Na₂HPO₄, 1 mM EDTA (pH 7.4) at 37° C. for 48 h. The formation of fibrils was confirmed by microscopy and binding of [³H]6-OH-BTA-1 ([³H]PIB). Fibrils were either used immediately or aliquoted and subsequently stored at −80° C. until use.

Solutions of the test substances (>95% purity according to analytical HPLC) or 6-OH-BTA-1 (>95% purity according to analytical HPLC; ABX Biochemicals, Radeberg, Germany) and N—[³H-methyl]6-OH-BTA-1 were prepared as 1-10 mM dimethyl sulfoxide (DMSO) stocks before dilution into assay buffer. The maximum final concentration of DMSO in the assays was 1%. All assays were performed in 10 mM Na₂HPO₄. The incubation was performed at 25° C. for 180 min. The bound and free fractions were separated by vacuum filtration through GF/B glass filters (Whatman, Maidstone, UK) using a Perkin Elmer harvester (PerkinElmer, 96 Micro B Filtermat) followed by 6×0.2 ml washes with ice cold phosphate buffer. Filters containing the bound ligand were counted with a liquid scintillation counter (Wallac Trilux, 1450 Microbeta).

The inhibition of [³H]PIB binding to fibrils of Aβ₄₀ and Aβ₄₂ was determined at 100 nM concentration of the test compound in question.

### Example 16

### Determination of inhibition constants Kᵢ of test compounds

The inhibition of [³H]PIB binding was measured for five different concentrations of the test compound in question, performed in duplicates. The determined IC₅₀ value was used for calculation of the inhibition constant, Kᵢ in the following manner

Kᵢ=IC₅₀/(1([L]/K_{σ}))

were [L] is the concentration (4 nM) and K_{d} (4.2 nM) of [³H]PIB used in the assay

### Example 17

### N-¹¹C-methylation of Primary and Secondary Amines

Cyclotron produced [¹¹C]CO₂ was converted to [¹¹C]CH₃I by the catalytic gas-phase iodination reaction via [¹¹C]CH₄ (GE MeI MicroLab). For further conversion to [¹¹C]CH₃OTf, the converted [¹¹C]CH₃I was distilled through a column of α-alumina impregnated with AgOTf.

10 μmmol of the amine compound in question was dissolved in anhydrous acetone (250 μl). The vial was sealed was flushed with and maintained under argon. The [¹¹C]CH₃OTf produced, swept with a He-flow at 50 ml/min, was trapped in the reaction vial at room temperature. The reaction vial was warmed to 65° C. over 30 s and kept at this temperature for 3 min, Thereafter the reaction mixture was diluted with 1 ml of a mixture of MeCN:0.1 M ammonium formate (ratio in the range of 27.5:72.5 to 40:60, v/v), loaded into a 2 ml injection loop and transferred onto a Chromolith C18 1 (ID: 10 mm; length: 100 mm; Merck). The column was eluted with a mobile phase consisting of MeCN:0.1 M ammonium formate (ratio in the range of 27.5:72.5 to 40:60, V/V) at a flow rate of 10 ml/min. In-line HPLC detectors included a UV detector (Sykam) set at 254 nm and a radioactivity detector (Bioscan Flow-Count fitted with a PIN detector). For animal experiments, the fraction containing the product was collected and diluted 1:1 with water. The mixture was applied to a Sep-Pak C18 and the cartridge subsequently washed with 10 ml water. The product was eluted with ethanol and diluted to the desired concentration with phosphate buffered saline. The pH of the final solution was between 7 and 8. Radiochemical purities were >95% as determined by analytical HPLC.

### Example 18

### O—₁₁C-methylation of Phenols

10 μmol of the phenolic compound was dissolved in anhydrous N′-N′-dimethyl-formamide (250 μl) containing 10 times molar excess of sodium hydride (NaH) and allowed to react for 5 min at ambient temperature. The solution was separated from the remaining NaH, transferred to a dry reaction vial. The vial was sealed, and subsequently flushed and maintained under argon. The [¹¹C]CH₃I produced, swept with a He-flow at 50 ml/min, was trapped in the reaction vial at ambient temperature. The reaction vial was warmed to 90° C. and kept at this temperature for 2-5 min. Thereafter the reaction mixture was diluted with 1 ml of MeCN:0.1 M ammonium formate (40:60, V/V), loaded into a 2 ml injection loop and transferred onto a Chromolith C18 1 (ID: 10 mm; length: 100 mm; Merck). The column was eluted with a mobile phase consisting of MeCN:0.1 M ammonium formate (40:60, v/v) at a flow rate of 10 ml/min. In-line HPLC detectors included a UV detector (Sykam) set at 254 nm and a γ-ray detector (Bioscan Flow-Count fitted with a PIN detector). For animal experiments, the fraction containing the product was collected and diluted 1:1 with water. The mixture was applied to a Sep-Pak C18 and the cartridge subsequently washed with 10 ml water. The product was eluted with ethanol and diluted to the desired concentration with phosphate buffered saline. The pH of the final solution was between 7 and 8. Radiochemical purities were >95% as determined by analytical HPLC.

### Example 19

### O-¹⁸F-fluoroethylation of Phenols

[¹⁸F]fluoride was produced through the ¹⁸O(p,n)¹⁸F nuclear reaction. The [¹⁸F]fluoride was obtained in a 34 mM solution of K₂CO₃ (0.3 ml) and added to a 2 ml conical vial containing 0.5 ml dry MeCN and 15 mg (39.9 μmol) Kryptofix 2.2.2. The solvent was evaporated with heating under reduced pressure. Azeotropic drying was repeated three times with 0.5 ml portions of MeCN. 2—[¹⁸F]-fluoroethyltosylate was prepared by dissolving 5 mg (12 μmol) of ethylene glycol-1,2-ditosylate in 250 μl MeCN and adding the solution to the dried kryptate ([K⊂2.2.2]⁺/¹⁸F⁻), the vial was then sealed and heated at 90° C. for 5 min. 2-[¹⁸F]-fluoroethyltosylate was purified by reversed phase HPLC (Lichrosorb C₁₈ 5μ; 10 mm×250 mm (CS-Chromatographie Service) eluted with MeOH/H₂O (50:50, volume/volume, flowrate 4 ml/min; k′=5.7; preparative radiochemical yield 55±5%). After on-line fixation of the product on a Strata X cartridge (33 μm; 30 mg/1 ml; Phenomenex) and drying of the product by argon-flush, the product was eluted with 0.15 ml DMF into a reaction vial.

10 μmmol of the phenolic compound in question was dissolved in anhydrous N′-N′-dimethyl-formamide (250 μl) containing 10 times molar excess of sodium hydride (NaH) and allowed to react for 5 min at ambient temperature. The solution was separated from the remaining NaH, transferred to a dry reaction vial. The vial was sealed was flushed with and maintained under argon. 2-[¹⁸F]fluoroethyl-tosylate was led into the reaction vial at room temperature. The reaction vial was warmed to 90° C. and kept at this temperature for 5 min. Thereafter the reaction mixture was diluted with 1 ml of MeCN:0.1 M ammonium formate (40:60, V/V), loaded into a 2 ml injection loop and transferred onto a Chromolith C18 (ID: 10 mm; length: 100 mm; Merck). The column was eluted with a mobile phase consisting of MeCN:0.1 M ammonium formate (40:60, v/v) at a flow rate of 10 ml/min. In-line HPLC detectors included a UV detector (Sykam) set at 254 nm and a radioactivity detector (Bioscan Flow-Count fitted with a PIN detector). For animal experiments, the fraction containing the product was collected and diluted 1:1 with water. The mixture was applied to a Sep-Pak C18 and the cartridge subsequently washed with 10 ml water. The product was eluted with ethanol and diluted to the desired concentration with phosphate buffered saline. The pH of the final solution was between 7 and 8. Radiochemical and chemical purities were >95% as determined by analytical HPLC.

### Example 20

### Preparation of ¹⁸F-fluorinated Compounds by Direct Fluorination

[¹⁸F]Fluoride was obtained in a 34 Mm solution of K₂CO₃ (0.3 ml) and added to a 2 ml conical vial containing 0.5 ml dry MeCN and 15 mg (39.9 Kryptofix 2.2.2. The solvent was evaporated with heating under reduced pressure. Azeotropic drying was repeated three times with 0.5 ml portions of MeCN. 10 μmol of the precursor in question was dissolved in 300 μl MeCN and adding the solution to the dried kryptate ([K⊂2.2.2]⁺/¹⁸F⁻), the vial was then sealed and heated at 90° C. for 5 min.

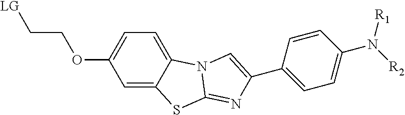

| | | | Temperature | Reaction | Yield^{a} |
|---|---|---|---|---|---|
| LG | R₁ | R₂ | (° C.) | time (min) | % |
| I | CH₃ | H | 90 | 5 | 32 |
| Br | CH₃ | H | 90 | 5 | 49 |
| OTs | CH₃ | H | 90 | 5 | 61 |
| Br | CH₃ | CH₃ | 90 | 5 | 48 |
| OTs | CH₃ | CH₃ | 90 | 5 | 58 |

^{a}Analytical yield.

### Example 21

### Preparation of ¹⁸F-fluorinated Compounds by Direct Fluorination

[¹⁸F]Fluoride was obtained in a 34 mM solution of K₂CO₃ (0.3 ml) and added to a 2 ml conical vial containing 0.5 ml dry MeCN and 15 mg (39.9 μmol) Kryptofix 2.2.2. The solvent was evaporated with heating under reduced pressure. Azeotropic drying was repeated three times with 0.5 ml portions of MeCN. 10 μmol of the precursor in question was dissolved in 300 μl MeCN and adding the solution to the dried kryptate ([K⊂2.2.2]⁺/¹⁸F⁻), the vial was then sealed and heated at a desired combination of temperature and reaction time, T

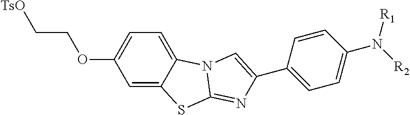

| | | Temperature | Reaction | Yield^{a} |
|---|---|---|---|---|
| R₁ | R₂ | (° C.) | time (min) | % |
| CH₃ | H | 80 | 5 | 32 |
| CH₃ | H | 90 | 2 | 28 |
| CH₃ | H | 90 | 5 | 49 |
| CH₃ | H | 90 | 10 | 57 |
| CH₃ | H | 90 | 15 | 54 |
| CH₃ | H | 100 | 2 | 34 |
| CH₃ | H | 100 | 5 | 52 |
| CH₃ | H | 100 | 10 | 48 |

^{a}Analytical yield.

### Example 22

### Preparation of ¹⁸F-fluorinated Compounds by Direct Fluorination

[¹⁸F]Fluoride was obtained in a 34 mM solution of K₂CO₃ (0.3 ml) and added to a 2 ml conical vial containing 0.5 ml dry MeCN and 15 mg (39.9 μmol) Kryptofix 2.2.2. The solvent was evaporated with heating under reduced pressure. Azeotropic drying was repeated three times with 0.5 ml portions of MeCN. 10 μmol of the tosyl-precursor in question was dissolved in the solvent of choice and added the solution to the dried kryptate ([K⊂2.2.2]⁺/¹⁸F⁻). Finally, the vial was then sealed and heated at 90° C. for 5 min.

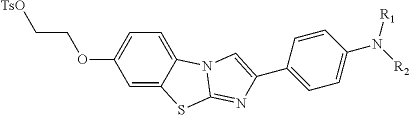

| | | Temperature | | Yield^{a} |
|---|---|---|---|---|
| R₁ | R₂ | (° C.) | Solvent | % |
| CH₃ | H | 90 | DMSO | 23 |
| CH₃ | H | 90 | DMF | 36 |
| CH₃ | H | 90 | DMAA | 42 |
| CH₃ | H | 90 | MeCN | 57 |

^{a}Analytical yield.

### Example 23

### Preparative ¹⁸F-radiosynthesis

[¹⁸F]Fluoride was obtained in a 34 mM solution of K₂CO₃ (0.3 ml) and added to a 2 ml conical vial containing 0.5 ml dry MeCN and 15 mg (39.9 μmol) Kryptofix 2.2.2. The solvent was evaporated with heating under reduced pressure. Azeotropic drying was repeated three times with 0.5 ml portions of MeCN. 10 μmol of the tosyl-precursor in question was dissolved in 300 μl MeCN and adding the solution to the dried kryptate ([K⊂2.2.2]⁺/¹⁸F⁻), the vial was then sealed and heated at 90° C. for 5 min. Thereafter the reaction mixture was diluted with 1 ml of a mixture of MeCN:0.1 M ammonium formate (ratio in the range of 27.5:72.5 to 40:60, v/v), loaded into a 2 ml injection loop and transferred onto a Chromolith C18 1 (ID: 10 mm; length: 100 mm; Merck). The column was eluted with a mobile phase consisting of MeCN:0.1 M ammonium formate (ratio in the range of 27.5:72.5 to 40:60, v/v) at a flow rate of 10 ml/min. In-line HPLC detectors included a UV detector (Sykam) set at 254 nm and a radioactivity detector (Bioscan Flow-Count fitted with a PIN detector). For animal experiments, the fraction containing the product was collected and diluted 1:1 with water. The mixture was applied to a Sep-Pak C18 and the cartridge subsequently washed with 10 ml water. The product was eluted with ethanol and diluted to the desired concentration with phosphate buffered saline. The pH of the final solution was between 7 and 8. Radiochemical purities were _{>}95% as determined by analytical HPLC.

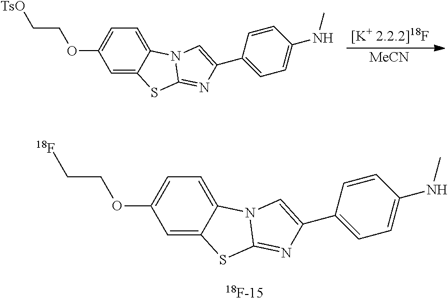

A typical applied amount of ¹⁸F-labeled tracer for the relevant studies is 2-3 mCi (74-111 MBq). This is based on the experience with a broad range of existing compounds for imaging of structures in the human central nervous system (CNS), including the four compounds that have been evaluated in humans for imaging of amyloid plaque in patients with Alzheimer's disease. The ¹⁸F-labeled compounds in question are prepared in a specific activity of >3000 mCi/μmol (111 GBq/μmol). Thus, an expected injected amount of the substances is <1×10⁻⁹ mol.

### Example 24

### Measurement of logP

Apparent drug lipophilicity was determined by a conventional partition method between 1-octanol and phosphate buffered saline (PBS), pH 7.4. The 1-octanol was saturated with PBS before use. Briefly, the no-carrier-added ¹¹C— or ¹⁸F-labeled compound in question, contained in 4 ml PBS, was added 4 ml of 1-octanol in a 20 ml test tube. The tube was sealed and vigorously shaken at room temperature for 2 min. The mixture was then centrifuged at 3000·g for 10 min. A 20 μl aliquot from each of the two phases were drawn and their radioactivity content was determined in a NaI(TI) well-type detector. The log P_{oct/PBS} was calculated as follows:

LogP_{oct/PBS}=log(radioactivity concentration in the 1-octanol phase I radioactivity concentration in the PBS phase). The reported values represent the mean of three independent measurements.

### Example 25

### Biodistribution Studies in Mice

The experiments were carried out with the approval of the institutional animal care committee (Regierung von Oberbayern, Munich, Germany) and in accordance with the German Animal Welfare Act (Deutsches Tierschutzgesetz). Animal husbandry followed the regulations of European Union (EU) guideline No. 86/609. Biodistribution studies were performed in male Balb-C mice (body weight of 19-25 g). The mice were injected in a lateral tail vein with 10-12 MBq of a high specific activity (>37 GBq/μmol) ¹¹C-labeled compound contained in 0.1-0.15 ml of a solution of isotonic phosphate buffered saline. Groups of mice were sacrificed at 5-30 min post-injection. The radioactivity of weighed tissue samples was measured in a γ-counter. Data are expressed as percent of the injected dose per gram tissue (% I.D./g; mean±sd, n=4−5).

### Example 26

### Speciation of Radioactive Compounds in Tissue of Mice

Male Balb-C mice were injected with 21-32 MBq of the compound of interest. At 10 and 30 min after injection the animals were sacrificed and the tissue of interest was dissected. The procedure used for the analysis of brain radioactivity was as follows: Brain tissue homogenates were prepared immediately after dissection by mechanical homogenization of nitrogen-frozen samples followed by addition of 1 ml of phosphate buffered saline. The mixture was vigorously vortexed, and 1 ml of MeCN was added. After centrifugation for 5 min at 6.000·g, the supernatant was collected. Approximately 0.1 ml of the supernatant solution was analyzed using radio-HPLC [Nucleosphere 100, 5 μm; 10×150 mm (CS-Chromatographie); MeCN/0.1 M ammonium formate 60:40, v/v]. The outlet of the column was connected in-line with a solid-phase scintillation counter.

The amount of intact tracer (Tᵢ) was calculated as follows:

Tᵢ=[F_{T}/F_{T}+F_{M}]×E_{E}×E_{R}×1·10⁻²

where F_{T} [%] represents the amount of intact tracer and F_{M} [%] the amount of metabolites as determined by radio-HPLC, corrected for extraction efficiency E_{E} [%] from the plasma samples and the recovery E_{R} [%] of activity from the HPLC. The extraction efficiency was in the range 68-92% among the compounds investigated.

### Example 27

### Investigation of In Vivo Regional Brain Uptake of Mice by Means of Small Animal PET

APP/PS1 transgenic mice (B6;CB-Tg(Thy1-PSEN1*M146V/Thy1-APP*swe)-10Arte) (Artemis Pharmaceuticals, Cologne, Germany) on a congenic C57BL/6J genetic background were used in the experiments. Anesthesia was begun 15 min ahead of experimental procedures by placing the animal in a cage ventilated with isoflurane (3%) and oxygen (3.5 l/min) with a pre-calibrated vaporizer. Anesthesia was maintained by 0.6% to 2% isoflurane and 3.5 l/min oxygen via a nose cone, such that the respiratory rate stayed at 80-100 per min. Body temperature was held at 37° C. with a temperature-controlled heating pad.

The transgenic animals were compared with commercially available age- and gender-matched controls (female mice by Harlan-Winkelmann, Borchen, Germany and male mice by Janvier, Le Genest-St-Isle, France), females weighing 26.1±2.0 g and males weighing 32.2±2.5 g.

All injections were performed under isoflurane inhalation anesthesia. An application catheter system for reliable intravenous access to the lateral tail veins was set up using 30-gauge needles, polythene tubing with 0.28 mm inner diameter, superglue and 1 ml syringes. To achieve long-term access an elastic hollow vessel-loop was used as a tourniquet. The catheter and syringe were initially filled with isotonic sodium chloride solution. The functional catheter was stabilized at the injection site with superglue.

All small-animal PET data were acquired with a microPET FOCUS F120 scanner (Siemens Medical Solutions, Malvern, USA) (Tai et al., 2005).

The tracer in question was applied in a volume of 124±73 μl injected via the catheter system intravenously in a slow bolus, followed by flushing with saline solution (84±26 μl isotonic sodium chloride solution 0.9%). Total applied volume was 253±76 μl. This approach provided that registered prompts at the end of application ranged between 150000 to 200000, ensuring a dead time<5% at 30 min p.i. Activities in the syringe were measured immediately before and after injection with a Capintech dose calibrator. Dynamic data acquisition was performed in 3D listmode starting immediately with injection of the tracer.

The emission data were normalized and corrected for the effects of scatter and attenuation. The resulting sinogram was reconstructed with FBP (filtered back-projection using a ramp filter with a cut-off at the Nyquist frequency) into 17 frames (5×1 min, 5×2 min, 5×5 min, 2×10 min). The image volume consisted of 128×128×95 voxels, with a size of 0.433×0.433×0.796 mm³ per voxel.

### Example 28

### Preparation of Mouse CNS for Ex Vivo Evaluation

Brain tissue was rapidly frozen in fine-crushed dry ice and stored air-tight at −75 C. For tissue analyses, every whole and half brain was mounted on a freezing medium and cut on a cryostat. The frozen sections were mounted on dilute poly-L-lysine hydrobromide coated (mol wt>300.000, (1:50) 0.01% w/v in water) microscopy slides. After drying at ambient conditions the remaining slides were stored at −75° C. until assayed.

### Example 29

### Thioflavin-T Staining

Deep frozen mouse brain sections were dried in ambient air for 15 min, immersion-fixed in 4% (w/v) paraformaldehyde (PFA) in phosphate buffered saline (PBS, pH=7.4) for 20 min at RT (20-22° C.). Sections were equilibrated in water twice for 2 min.

Thioflavin T (ThT) was dissolved at 1% (w/v) in water, and the solution was filtered. Sections were immersed in 1% ThT for 30 min at RT and kept dark, rinsed for 3 min in water, and differentiated in two changes of 80% ethanol (5 min and 1 min), washed again in three changes of water (2 min each) and mounted in ProLong Gold antifade mounting medium (Invitrogen, Karlsruhe, Germany) with coverslips.

### Example 30

### Immunofluorescence for Aβ₄₀ and Aβ₄₂

Immunofluorescence stainings of amyloid plaques were carried out selectively for Aβ₄₀ and Aβ₄₂ components. For stainings, sections were prepared with 70% formic acid for 20 min and washed four times. Then, they were blocked with 3% bovine serum albumin in PBS for 15 min and then probed with the two primary antibodies (G2-13 (The Genetics, Schlieren, Switzerland)) and AB2 (Araclon Biotech, Zaragoza, Spain)) diluted in 1% BSA/PBS over night at 4° C., washed with PBS, blocked again with 3% BSA/PBS and incubated with two fluorophore-conjugated secondary antibodies (A488-D-Rb (Invitrogen, Karlsruhe, Germany) and Cy5-D-Ms (Jackson ImmunoResearch, Suffolk, Great Britain). After a short wash, nuclei were stained by adding 0.5 μM DAPI for 3 min. After the final washing steps the tissue was coverslip-mounted with ProLong Gold Antifade mounting medium.

### Example 31

### Ex Vivo Analysis of Regional Distribution in Brain by Autoradiography

Digital autoradiographic images with a field of view of 24×32 mm were taken with the M40 series of μ-Imager™ (Biospace lab, Paris, France) using 10×10 cm scintillating foils with 13±1.5 μm thickness (Applied Scintillation T Administration of [³H]PIB and radiolabeled test compounds for autoradiography

For ex vivo autoradiographical assessment the radiolabeled tracer was coinjected with [³H]PIB (specific activity: 2.78 TBq/mmol, radiochemical purity>97%) into live animals.

The animals were killed and the full brain was removed within 8±2 min post-mortem, rapidly frozen in fine-crushed dry ice prior to sectioning.

echnologies, Harlow, England). The resolution with tritium is 20 μm, the detection threshold for tritium is 0.4 cpm/mm² and the smallest pixel size is 1 μm.

Instrument acquisition was controlled with μ-Acquisition software. The processing, quantitation and data management was handled with β-Vision+software (both by Biospace lab).

Deep frozen CNS and whole head sections were dried in ambient air for 30 min and covered with the scintillation foil. Three full axial sections were acquired in one scan. An optical image was taken first. A total of 3 million counts were obtained for each sample.

### Example 32

### Fluorescence Microscopy

Fluorescence microscopy was performed with an Axiolmager Z.1 microscope (Carl Zeiss, Göttingen, Germany). Entire-view micrographs of axial whole mouse brain sections were is recorded through a 10′10.3 EC Plan Neofluar Zeiss lens and Zeiss filter sets no. 49 (DAPI), no. 38 (HE Green Fluorescent Protein), no. 43 (HE DsRed), no. 47 (HE Cyan Fluorescent Protein) and no. 50 (Cy5). For the entire view, MosaiX pictures were created with a Zeiss CAN-Bus motor stage (Merzhäuser, Germany) and digitalized with an AxioCam MRm Rev. 3.0 (Carl Zeiss). AxioVision software, release 4.6, was used for acquisition and processing. The single micrographs were stitched to full-section view with the AxioVision MosaiX module.

### Example 33

### Processing of μPET Data

All raw PET data sets were loaded into Amide 0.8.22 software. Each PET data set was cropped to contain the head and neck only. PET original data and scale formats (32-bit float with per plane scale factor and 16-bit unsigned short with single scale factor, respectively) were preserved for the cropped output. The data sets were worked on in tri-linear interpolation type.

### Example 34

### Inhibition of [³H]Binding to AD Post-Mortem Brain Tissue

AD post-mortem brain tissue was obtained from the Brain Bank Germany. The neuropathological diagnosis was confirmed by current criteria (Break stage V1). The presence and localization of plaques on the sections was confirmed with immunohistochemical staining with fluorescent monoclonal anti-Aβ antibodies: A4 (DAB) and (Apaap). The frozen sections were incubated with [³H]PIB (1-2×10⁶ cpm/ml) for 1 h at room temperature with and without a test compound present at 100 nM concentration.

1-18. (canceled)

19. A tracer for aggregates of amyloid peptides comprising the structure 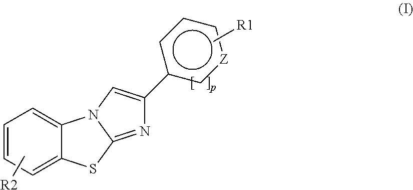
wherein R₁ is selected from the group consisting of H, F, Cl, Br, I, CN, CF₃, alkyl, heteroaryl, heterocycloalkyl and NHR₃, with R₃ being an alkyl,
R₂ is —O—R₄, wherein R₄ is selected from the group consisting of CₙH₂ₙ+1, CₙH₂ₙ, CₙH₂ₙ-halo, —CH₂—CH═CH-halo, and —[CH₂—CH₂—O]ₘ,—[CH₂—CH₂]ₒ-halo, in which halo can be any halogen, and with n being in the range of from 1 to 5, with m being in the range of from 1 to 3 and with o being 1, and
wherein Z is selected from the group consisting of S, O, N, NH and CH, and
wherein p is 0 or 1, wherein the tracer comprises at least one F.

20. The tracer of claim 19, wherein R₂ is selected from the group consisting of —O—CₙH₂ₙ—F, —O—CH₂—CH═CH—F and —O—[CH₂—CH₂—O]ₘ[CH₂—CH₂]ₒ—F.

21. The tracer of claim 19, wherein R₂ is selected from the group consisting of —O—CH₂CH₂—F, —O—CH₂CH₂CH₂—F, —O—CH₂CH₂—O—CH₂CH₂—F and —O—CH₂CH₂-β-CH₂CH₂—O—CH₂CH₂—F.

22. The tracer of claim 19, comprising the structure 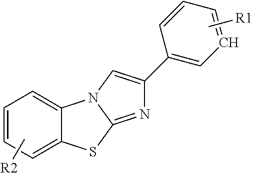
wherein R₁ is selected from the group consisting of NHR₃, an optionally substituted nitrogen containing heteroaryl and an optionally substituted nitrogen containing heterocycloalkyl.

23. The tracer of claim 22, wherein R₁ is NHR₃.

24. The tracer of claim 23, having a structure selected from the group consisting of:

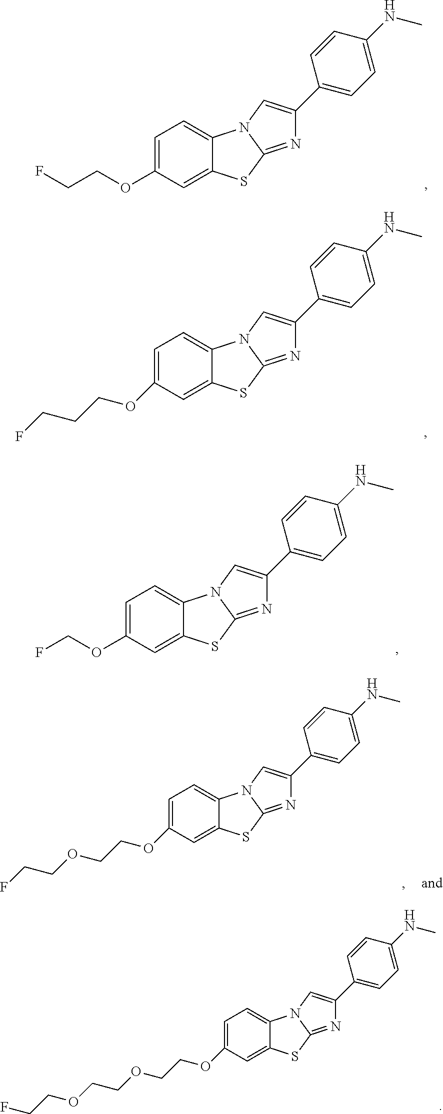

25. The tracer of claim 22, wherein R₁ is an optionally substituted nitrogen containing heterocycle or an optionally substituted nitrogen containing heteroaryl.

26. The tracer of claim 19, comprising the structure

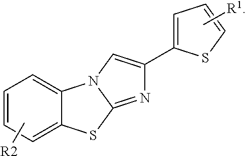

27. The tracer of claim 26, wherein the tracer has a structure selected from the group consisting of:

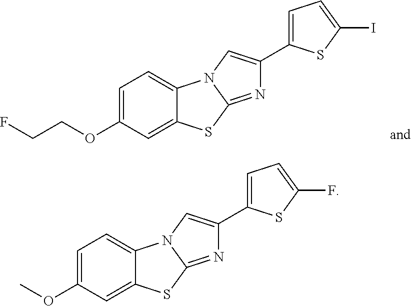

28. A method for the preparation of a tracer having the general formula 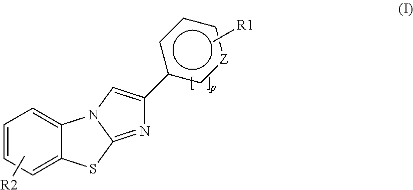
wherein R₁ is selected from the group consisting of H, F, Cl, Br, I, CN, CF₃, alkyl, heteroaryl, heterocycloalkyl and NHR₃, with R₃ being an alkyl, wherein R₂ is selected from the group consisting of —O—CₙH₂ₙ—F, —O—CH₂—CH═CH—F and —O—[CH₂—CH₂—O]ₘ—[CH₂—CH₂]ₒ—F, and wherein Z is selected from the group consisting of S, O, N, NH and CH, and wherein p is 0 or 1, the method comprising fluorinating a derivative of the general formula II 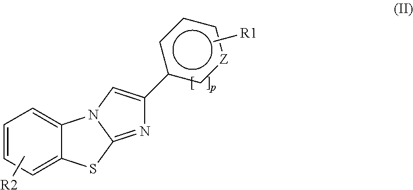
wherein R₁ is selected from the group consisting of H, F, Cl, Br, I, CN, CF₃, alkyl, heteroaryl, heterocycloalkyl and NHR₃, with R₃ being an alkyl, wherein R₂ is selected from the group consisting of —O—CₙH₂ₙ-leaving group, —O—CH₂—CH═CH-leaving group and —O—[CH₂—CH₂—O]ₘ—[CH₂—CH₂]ₒ— leaving group, and wherein Z is selected from the group consisting of S, O, N, NH and CH, and wherein p is 0 or 1,
with [¹⁸F]fluoride.

29. The method of claim 28, wherein the leaving group is selected from the group consisting of halogen atoms, tosylsulfonate, trifluoromethanesulfonate, tolylsulfonatetriflate, tosylate, trialkyl ammonium, nitro and azide.

30. The method of claim 28, wherein said fluorinating is performed in a solvent.)

31. The method of claim 30, wherein the solvent is selected from the group consisting of dimethylformamide (DMF), tetrahydrofurane (THF), dimethylsulfoxide (DMSO), acetonitrile and acetamide.

32. The method of claim 28, wherein said fluorinating is performed under microwave heating.

33. The method of claim 28, wherein said fluorinating is performed at a temperature in the range of 0.25-200° C.

34. The method of claim 28, wherein a ratio of [¹⁸F]fluoride to the precursor derivative is in the range of 1:1,000 to 1:100,000.

35. The tracer of claim 19, wherein at least one of R₁ or R₂, independently comprises ¹¹C_{,} ¹⁸F, ¹⁹F, ⁷⁵Br, ⁷⁶Br, ¹²³I, ¹³¹I, or ¹²⁴I.

36. A method of imaging and quantifying amyloidosis in Alzheimer's disease, type II diabetes, Down's syndrome, Creutzfeldt-Jacob disease, prion mediated diseases, amyloid polyneuropathy or amyloid cardiomyopathy, using the tracer of claim 19.

37. A method of preparing a diagnostic compound for the imaging and quantification of amyloidosis in Alzheimer's disease, type II diabetes, Down's syndrome, Creutzfeldt-Jacob disease, prion mediated diseases, amyloid polyneuropathy or amyloid cardiomyopathy, using the tracer of claim 19.

38. The tracer of claim 20, wherein F is ¹⁸F.

39. The tracer of claim 21, wherein R₂ is —O—CH₂CH₂—F.

40. The tracer of claim 23, wherein R₃ is NHMe.

41. The tracer of claim 24, having the following structure:

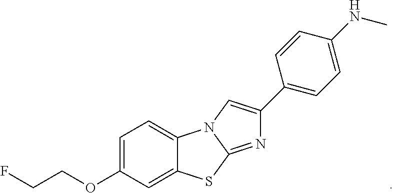

42. The tracer of claim 25, wherein the tracer has a structure selected from the group consisting of

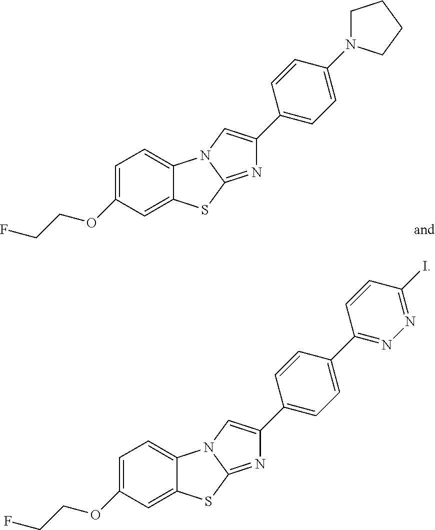

43. The tracer of claim 26, wherein R₁ is selected from the group consisting of H, F, Cl, Br, I, CN, CF₃, and alkyl.
